(19) United States
(12) Patent Application Publication
(10) Pub. No.: US 2024/0241239 A1
(43) Pub. Date: Jul. 18, 2024

(54) SINGLE-SHOT 3D IMAGING USING A SINGLE DETECTOR

(71) Applicant: California Institute of Technology, Pasadena, CA (US)

(72) Inventors: Lihong Wang, Arcadia, CA (US); Yide Zhang, Pasadena, CA (US)

(21) Appl. No.: 18/410,842

(22) Filed: Jan. 11, 2024

Related U.S. Application Data
(60) Provisional application No. 63/438,654, filed on Jan. 12, 2023.

(51) Int. Cl. G01S 7/52; A61B 5/00; A61B 8/00; G01S 15/89; H02N 2/00

(52) U.S. Cl. CPC G01S 7/52079; A61B 5/0095; A61B 8/4427; A61B 8/4483; G01S 15/8965; G01S 15/8993; H02N 2/22

(57) ABSTRACT

Among the various aspects of the present disclosure is the provision of systems and methods of imaging using photoacoustic computed tomography through an ergodic relay having an integrated single-element ultrasonic transducer.

### CROSS-REFERENCES TO RELATED APPLICATIONS

This application claims benefit of and priority to U.S. Provisional Patent Application No. 63/438,644, titled “SINGLE-SHOT 3D IMAGING USING A SINGLE DETECTOR,” and filed on Jan. 12, 2023, which is incorporated by reference herein in its entirety and for all purposes.

### FEDERALLY SPONSORED RESEARCH OR DEVELOPMENT

This invention was made with government support under Grant No(s). EB028277 & CA220436 & EB029823 awarded by the National Institutes of Health. The government has certain rights in the invention.

### FIELD

Certain aspects generally pertain to photoacoustic imaging and, more specifically, to 3D photoacoustic imaging systems, devices, and methods.

### BACKGROUND

Vascular diseases, such as atherosclerosis, thrombosis, aneurysms, and peripheral vascular diseases, pose serious health risks such as heart attack, stroke, organ failure, and complications in the lower extremities. Imaging of hemodynamics, especially monitoring the blood supply to lower extremities is a valuable tool in early detection and intervention in these diseases. Moreover, measuring factors like blood flow velocity can also evaluate an individual's risk of developing vascular diseases, aiding preventive measures and facilitating personalized treatment plans.

### SUMMARY

Certain embodiments pertain to an ergodic relay comprising an acoustic delay line (e.g., an optical rod or acoustic waveguide) configured to temporally separate one or more initial photoacoustic signals and one or more reflected photoacoustic signals. The ergodic relay further comprises an acoustic cavity (e.g., glass prism) coupled to the acoustic delay line and an integrated single-element ultrasonic transducer fabricated onto a surface of the acoustic cavity.

Certain embodiments pertain to a photoacoustic computed tomography system comprising a housing and an ergodic relay at least partially located within the housing. The ergodic relay comprising an acoustic delay line configured to temporally separate one or more initial photoacoustic signals and one or more reflected photoacoustic signals, an acoustic cavity coupled to the acoustic delay line, and an integrated single-element ultrasonic transducer fabricated onto a surface of the acoustic cavity. The photoacoustic computed tomography system further comprising one or more optical fibers coupled to the acoustic cavity, wherein the one or more optical fibers are configured to receive an illumination beam. In one case, the photoacoustic computed tomography system is a handheld device.

Certain embodiments pertain to photoacoustic computed tomography imaging methods. In some cases, a method receives encoded photoacoustic data based on photoacoustic signals detected by an integrated single-element ultrasonic transducer fabricated directly onto a surface of a prism of an ergodic relay. The ergodic relay includes an optical rod fused to the prism. The optical rod is configured to temporally separate the photoacoustic signals and the reflected photoacoustic signals. The method also reconstructs one or more 3D photoacoustic images from the photoacoustic data using a plurality of calibrated virtual transducers.

These and other features and embodiments will be described in more detail with reference to the drawings.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIG. 1 depicts a block diagram of a PACTER system in an imaging configuration, according to various embodiments.

FIG. 2 depicts a block diagram of a PACTER system in a calibration configuration, according to various embodiments.

FIG. 3 depicts a schematic diagram of a PACTER system in a calibration configuration, according to an embodiment.

FIG. 4 depicts a schematic diagram of PACTER system in an imaging configuration, according to an embodiment.

FIG. 5A depicts a schematic diagram of an ergodic relay 550, according to an embodiment.

FIG. 5B depicts graphs of 1D(t) PACTER signals detected by single-element ultrasonic transducer in FIG. 5A at time instances t₀, t₁, . . . t_{N} and a 4D (xyzt) reconstructed image of human palmar vessels from the 1D(t) PACTER signals, according to an embodiment.

FIG. 6A depicts a schematic illustration of an ergodic relay of a PACTER system in a calibration configuration, according to an embodiment.

FIG. 6B depicts a schematic illustration of ergodic relay of PACTER system in FIG. 6A in an imaging configuration in which palmar vessels of a human hand are being imaged, according to an embodiment.

FIG. 7A depicts plots of PACTER calibration signals of the calibrated virtual transducers corresponding to r₁, r₂, and r₃ in FIG. 6A.

FIG. 7B depicts plots of PACTER from source points r′₁ and r′₂, according to an embodiment.

FIG. 8A depicts a 3D PACTER image of a curved black wire acquired by the PACTER system in FIG. 4, according to an embodiment.

FIG. 8B depicts snapshots taken by PACTER system in FIG. 4 at time instances t₀=1.15s, t₁=2.25s, . . . t_{N}=3.85s showing bovine blood flushing through an S-shaped tube, according to an embodiment.

FIG. 8C depict 3D PACTER images taken by PACTER system in FIG. 4 of the three bars printed with black ink on a transparent film, according to an embodiment.

FIG. 9A depicts a plot of the PACTER reconstructed z-positions versus real (measured) z positions of the three bars in FIG. 8C, according to an embodiment.

FIG. 9B depicts a 3D PACTER image of the two crossing human hairs in agarose taken by PACTER system, according to an embodiment.

FIG. 9C depicts two PACTER images showing the crossing pattern, according to an embodiment.

FIG. 10A depicts profiles along the dashed lines in FIG. 9C, according to an embodiment.

FIG. 10B depicts a 3D PACTER image of bovine blood flushing through a tube as imaged by PACTER system in FIG. 4, according to an embodiment.

FIG. 10C depicts photoacoustic amplitudes along the tube in FIG. 10B versus time, when the blood flushes through the tube at different speeds, according to an embodiment.

FIG. 10D depicts a plot of the speeds of the blood flow quantified from the reconstructed PACTER images in FIGS. 28A-I versus the real speeds of the blood flow set by the syringe pump along the tube shown in FIG. 10B, according to an embodiment.

FIG. 11A depicts a schematic illustration of a mouse PACTER imaging configuration where the mouse abdomen is located directly on a distal end of an optical rod of an ergodic relay of a PACTER system, according to an embodiment.

FIG. 11B depicts a 3D PACTER image of the abdominal vasculature of a first mouse captured by PACTER system in FIG. 4, according to an embodiment.

FIG. 11C depicts a 3D PACTER image of the abdominal vasculature of a second mouse captured by PACTER system in FIG. 4, according to an embodiment.

FIG. 11D depicts cross-sectional 2D images of mouse 1 at four different time instances and a plot of photoacoustic amplitudes versus time, according to an embodiment.

FIG. 11E depicts cross-sectional 2D images corresponding to the rectangle in FIG. 11C of mouse 2 at four different time instances and a plot of the photoacoustic amplitudes versus time, according to an embodiment.

FIG. 12A depicts a plot of the center positions and widths of vessels versus time, based on fits from the cross-sectional 2D images of FIG. 11D, according to an embodiment.

FIG. 12B depicts a plot of the center positions and widths of vessels versus time based on cross-sectional 2D images of FIG. 11D, according to an embodiment.

FIG. 12C depicts a plot of Fourier transforms of center positions and widths of the vessels in FIG. 12A, according to an embodiment.

FIG. 12D depicts a plot of Fourier transforms of center positions and widths of the vessels in FIG. 12B showing respiratory frequency, according to an embodiment.

FIG. 13A depicts a schematic illustration of a human hand PACTER imaging configuration, according to an embodiment.

FIG. 13B depicts a 3D PACTER image of thenar vasculature, according to an embodiment.

FIG. 13C depicts a 3D PACTER image of the thenar vasculature, according to an embodiment.

FIG. 14A depicts the maximum amplitude projections of the 3D volumes from the 4D PACTER datasets in FIG. 13B, according to an embodiment.

FIG. 14B depicts the maximum amplitude projections of 3D volumes from the 4D PACTER datasets in FIG. 13C for Subject 2 before, during, and after cuffing, according to an embodiment.

FIG. 15A depicts a plot of the positions of the blood front along the blood vessel during the occlusion and recovery phases in FIG. 14A, according to an embodiment.

FIG. 15B depicts a plot of the comparison between the durations of the occlusion and recovery phases in FIG. 14B, according to an embodiment.

FIG. 15C depicts a plot of positions of the blood front along the blood vessel during the occlusion and recovery phases in FIG. 14B, according to an embodiment.

FIG. 15D depicts a bar graph of a comparison between the durations of the occlusion and recovery phases in FIG. 14B, according to an embodiment.

FIG. 15E depicts a bar graph of a comparison between the blood flow speeds during recovery in FIGS. 14A and 14B, according to an embodiment.

FIG. 16A is a schematic illustration of a human foot PACTER imaging configuration, according to an embodiment.

FIG. 16B depicts a 3D PACTER image of the foot vessels of the subject before vascular occlusion, according to an embodiment.

FIG. 16C depicts a 3D PACTER image of the foot vessels of the subject after vascular occlusion, according to an embodiment.

FIG. 17A depicts maximum amplitude projections of the 3D volumes from the 4D PACTER datasets in FIG. 16B, according to an embodiment.

FIG. 17B depicts an illustration of a difference between the two images in FIG. 17A, according to an embodiment.

FIG. 17C depicts a plot of relative photoacoustic signals of venous and arterial regions indicated by circles in FIGS. 17A and 17B respectively.

FIG. 18 depicts a PACTER images of a middle finger, an index finger, a proximal phalanx region, and a thenar region, according to an embodiment.

FIG. 19 depicts a flowchart of operations of a method of fabricating an ergodic relay with an integrated single-element ultrasonic transducer, according to an embodiment.

FIG. 20 depicts a flowchart of a PACTER method of calibrating a PACTER system, according to an embodiment.

FIG. 21 depicts a flowchart of a PACTER imaging using a PACTER system such as PACTER system in FIG. 1, according to an embodiment.

FIG. 22A depicts a schematic illustration of the working principle of a fly's eye homogenizer having a microlens array, according to an embodiment.

FIG. 22B depicts a first photograph of the spatial distribution of an original laser beam from a high power laser source and a second photograph of a homogenized beam.

FIG. 22C depicts a schematic illustration of an example of an optical path light through a fly's eye homogenizer and an ergodic relay of a PACTER system, according to an embodiment.

FIG. 23A depicts photographs showing the PACTER imaging setup of a black wire when the illumination area is smaller, equivalent, and larger compared with the calibrated area, according to an embodiment.

FIG. 23B depicts 3D PACTER images of the black wire in FIG. 23A acquired when the illumination area is smaller, equivalent, and larger compared with the calibrated area, according to an embodiment.

FIG. 23C depicts a schematic drawing of a view an ergodic relay with an integrated single-element ultrasonic transducer, according to an embodiment.

FIG. 23D depicts a schematic drawing of another view of ergodic relay in FIG. 23C.

FIG. 23E depicts a schematic drawing of another view of ergodic relay in FIG. 23C.

FIG. 24 is a schematic representation of sputter fabrication of material layers of an integrated single-element ultrasonic transducer 2460 onto a hypotenuse surface of a prism 2452 of an ergodic relay 2450, according to an embodiment.

FIG. 25A depicts a schematic drawing of an example of an optical path of an ergodic relay of a PACTER system, according to an embodiment.

FIG. 25B is a reconstructed image of a black wire using a PACTER system calibrated with bovine blood, showing object independence or universality of calibration, according to an embodiment.

FIG. 25C depicts a time-lapse simulation of a PA signal propagating in the ergodic relay in the PACTER system in FIG. 25A and a plot of the PA signal, according to an embodiment.

FIG. 26 depicts reconstructed PACTER images using calibration data acquired at different times over the course of a year with the PACTER system in FIG. 3, according to an embodiment.

FIG. 27A depicts a 3D PACTER image of three bars printed with black ink on a transparent film, according to an embodiment.

FIG. 27B depicts a summation of the maximum y-projections of the 3D volumes like FIG. 27A where the object is placed at different depths, according to an embodiment.

FIG. 27C depicts a plot of profiles along the solid lines in FIG. 27B, according to an embodiment.

FIG. 28A depicts a first image of PA amplitudes along a tube flushed by the blood with a speed of 38.9 mm/s versus time, according to an embodiment.

FIG. 28B depicts a second differential image showing the distance the blood front travels over time, according to an embodiment.

FIG. 28C depicts a plot of traveling distance of the blood front plotted over time, according to an embodiment.

FIG. 28D depicts an image of PA amplitudes along the tube flushed by the blood with different speeds versus time, according to an embodiment.

FIG. 28E depicts an image of PA amplitudes along the tube flushed by the blood with different speeds versus time, according to an embodiment.

FIG. 28F depicts an image of PA amplitudes along the tube flushed by the blood with different speeds versus time, according to an embodiment.

FIG. 28G depicts an image of PA amplitudes along the tube flushed by the blood with different speeds versus time, according to an embodiment.

FIG. 28H depicts an image of PA amplitudes along the tube flushed by the blood with different speeds versus time, according to an embodiment.

FIG. 28I depicts an image of PA amplitudes along the tube flushed by the blood with different speeds versus time, according to an embodiment.

FIG. 29 is a schematic drawing of a PACTER system configured for handheld imaging operation, according to an embodiment.

FIG. 30A is a schematic drawing of a PACTER system with a temperature control system, according to an embodiment.

FIG. 30B is a schematic drawing of a PACTER system for showing the temperature dependence, according to an embodiment.

FIG. 31A depicts a graph of a correlation coefficient, according to an embodiment.

FIG. 31B depicts a plot of a PACTER signal acquired with heating, according to an embodiment.

FIG. 31C depicts a plot of a PACTER signal acquired at 1300 s with heating off, according to an embodiment.

FIG. 31D depicts a plot of a PACTER signal acquired at 2410 s with heating on, according to an embodiment.

FIG. 32 depicts an ergodic relay showing the dimensions, according to an embodiment.

FIG. 33A depicts a schematic diagram of an ergodic relay in a PACTER system showing a simulated PA signal propagating in the ergodic relay, according to an embodiment.

FIG. 33B depicts a schematic diagram of ergodic relay with an acoustic impedance-matching layer, according to an embodiment.

FIG. 34A depicts a schematic drawing of an ultrasonography system with a transmitter and a receiver array with hundreds of elements.

FIG. 34B depicts a schematic drawing of ultrasonography PACTER system, according to an embodiment.

The figures and components therein may not be drawn to scale.

### DETAILED DESCRIPTION

Different aspects are described below with reference to the accompanying drawings. The features illustrated in the drawings may not be to scale. In the following description, numerous specific details are set forth in order to provide a thorough understanding of the presented embodiments. The disclosed embodiments may be practiced without one or more of these specific details. In other instances, well-known operations have not been described in detail to avoid unnecessarily obscuring the disclosed embodiments. While the disclosed embodiments will be described in conjunction with the specific embodiments, it will be understood that it is not intended to limit the disclosed embodiments.

### I. Introduction

### (A) Photoacoustic Computed Tomography Through an Ergodic Relay (PACTER) Techniques

3D imaging methods can be generally classified into two categories. The first category includes, e.g., photoacoustic microscopy, confocal microscopy, two-photon microscopy, and radar, and requires the sequentially scanning of the probing beam across the object. This first category of 3D imaging methods uses single- or few-element detectors but suffers a low imaging speed due to a required sequential scanning scheme. The second category includes, e.g., photoacoustic computed tomography, light-sheet microscopy, ultrasonography, and sonar, and can capture a 3D image of an object using one or a few exposures of the probing beam. However, the methods of this second category usually require multiple detectors or multi-element detector arrays, which are often complex, expensive, bulky, and not particularly suitable for portable or wearable applications. These problems may thus limit the translation of these 3D imaging methods that can be used for biomedical research, such as photoacoustic computed tomography, to clinical settings.

Certain embodiments pertain to photoacoustic computed tomography through an ergodic relay (PACTER) methods, systems, and devices. Certain PACTER techniques can acquire a 3D image of an object with a single exposure using a single-element ultrasonic detector that can function as multiple virtual detectors (e.g., thousands of virtual detectors). In some cases, the single-element element detector is an integrated detector fabricated directly onto a surface of the ergodic relay. Distinct from other 3D imaging methods, PACTER techniques can provide a 3D image of an object with a single exposure of a probing beam, using a single-element ultrasonic detector. This single-shot imaging capability may provide faster imaging speeds as compared to other methods that require sequential scanning, such as photoacoustic microscopy, confocal microscopy, two-photon microscopy, and radar. In one aspect, a PACTER system can provide up to a 1 kHz volumetric imaging speed. In another aspect, a PACTER system using a laser with a high repetition rate such as a repetition rate of 1 MHz can have a volumetric imaging speed of 1 MHz. The single element ultrasonic detector may also significantly reduce system complexity, cost, and form factor as compared with other 3D imaging methods that use multiple(-element) detectors such as light-sheet microscopy, ultrasonography, and sonar. Also, certain PACTER techniques do not require re-calibration for different objects.

Certain embodiments are directed to an ergodic relay (ER) with an acoustic cavity such as a glass prism, an acoustic delay line such as an optical rod (e.g., glass rod) or an acoustic waveguide, and a single element transducer element. In some cases, the single-element transducer element is an integrated component of the ergodic relay fabricated directly onto a surface of the acoustic cavity. In one embodiment, the ergodic relay includes a glass prism fused to a silica optical rod and an integrated ultrasonic transducer based on a lead magnesium niobate-lead titanate (PMN-PT) single crystal. In other embodiments, alternative materials may be used to fabricate the ergodic relay and/or the transducer, which may provide improved 3D single-shot imaging performance. For example, the acoustic cavity can be fabricated with other transparent materials including, but not limited to, polymethyl methacrylate, epoxy, and polycarbonate. As another example, the ultrasonic transducer can be implemented with other materials, including, but not limited to, lead zirconium titanate, lithium niobate, and polyvinylidene fluoride.

Alternatively, or additionally, the shapes and dimensions of the ergodic relay in the PACTER devices and systems can be variated. In one example, the ergodic relay may include a glass rod that functions as an acoustic delay line to temporally separate the initial photoacoustic signals and the reflected photoacoustic signals. In alternative embodiments, the glass rod may be replaced with an acoustic waveguide with different designs. In another example, the ergodic relay may include a prism as an acoustic cavity for scrambling or encoding acoustic waves. In alternative embodiments, the prism be replaced with any type of acoustic cavity that can scramble the acoustic waves such as, e.g., glass/quartz plates (e.g., slides, coverslips), silicon wafers, etc. Alternatively, the ergodic relay can be a whole piece of material that functioning as both the acoustic delay line and the acoustic cavity.

In various embodiments, the PACTER imaging procedure uses photoacoustic signals from a single-element transducer element. Although many embodiments of PACTER systems include only a single transducer element, it would be understood that one or more additional transducer elements may be included in other implementations for, e.g., redundancy.

As provided in Section IV, PACTER techniques have been demonstrated to image vital signs in small animals and 3D hemodynamics in humans. Possible applications of PACTER techniques include, but are not limited to, biometrics, home-care monitoring (home care of diabetic-foot ulcer or carotid-artery disease), point-of-care testing (point-of-care screening for hypertension), and noninvasive hemodynamic monitoring (simultaneous oximetry of both arterial and venous blood) in intensive care units.

Other possible applications of the single-detector technique in PACTER that implements a single-element detector to function as potentially thousands of virtual detectors are other imaging technologies including, but not limited to, medical ultrasonography, underwater sonar, and airborne radar.

### (B) Imaging Hemodynamics Using PACTER Techniques

There are several techniques available to image hemodynamics in the human body, each with its own strengths and limitations. Magnetic resonance imaging (MRI), computed tomography (CT) angiography, and positron emission tomography (PET) are all capable of producing high-resolution images of the vascular system and blood flow dynamics, but they require the use of ionizing radiation and the injection of contrast agents, which can have adverse health effects. Moreover, relying on strong ionizing sources and numerous detector elements, these techniques are bulky and expensive, making them inaccessible to mobile clinics or small healthcare facilities. Optical imaging techniques, such as fluorescence imaging and optical coherence tomography (OCT), offer noninvasive visualization of hemodynamics, but their penetration depths are constrained by the optical diffusion limit (˜1-2 mm) and do not have sufficient specificity to hemoglobin. However, even with recent improvements in minimizing ultrasound probes, state-of-the-art ultrasound imaging techniques still require burdensome and costly data acquisition systems due to hundreds to thousands of detector elements.

Photoacoustic tomography (PAT), also referred to as optoacoustic tomography, offers a promising solution to the limitations faced by other imaging techniques for hemodynamic imaging. Unlike other techniques, PAT utilizes the photoacoustic (PA) effect to absorb the energy of incident photons by optical absorbers, such as hemoglobin, in biological tissue and re-emit them as ultrasonic waves (PA waves) to generate optical contrast tomographic images. As a result, PAT does not rely on ionizing radiation or contrast agents. Moreover, due to the weak scattering of ultrasound in biological tissue, PAT provides a depth-to-resolution ratio of approximately 200, enabling high spatial resolution at depths up to several centimeters. Two primary forms of PAT are photoacoustic microscopy (PAM) and photoacoustic computed tomography (PACT). PAM requires sequential scanning of the probing beam and PACT captures a 3D image using one or a few pulses of the probing beam. PAM utilizes a single-element detector, which requires a simple data acquisition system but suffers from low imaging speed. In contrast, while PACT can offer higher imaging speeds of up to kilohertz, PACT generally necessitates a number of detection elements and corresponding data acquisition systems. This results in systems that are complex, costly, and bulky.

Some PACT systems have a reduced number of detector elements. For example, one PACT approach utilizes the principles of compressive sensing and single-pixel imaging. This approach uses acoustic scatterers to achieve PA or ultrasound tomography with just a single detector element. However, these PACT techniques are time-consuming, as they require a sequence of measurements with different mask configurations, limiting their speed. To try to address this issue, researchers have developed methods that take advantage of the spatiotemporal encoding of an ergodic relay (ER) or a chaotic cavity. These techniques can produce single-shot images while using fewer detector elements. However, these PACT systems only perform 2D imaging and require recalibration for different objects, which can be time-consuming. Additionally, they may not be suitable for long-term imaging in unstable environments due to their sensitivity to boundary conditions. As discussed above, other techniques for imaging hemodynamics use ionizing radiation or contrast agents, or are limited to imaging depths within approximately 1 millimeter, by complex and expensive data-acquisition systems, or by low imaging speeds, complexity, or cost.

Certain embodiments described herein pertain to PACTER techniques that implement photoacoustic tomography using a single laser pulse and a single transducer element that can function as thousands of virtual detectors, which allows for the volumetric capture of fast hemodynamic changes in, for example, human feet. Certain embodiments described herein pertain to PACTER techniques that can have ultrafast volumetric photoacoustic imaging of hemodynamics in the human body where high imaging rate (e.g., up to 1 MHz when using high repetition rate laser of 1 MHz can be achieved by using a single laser pulse and a single element functioning a plurality of detectors (e.g., 6,400 virtual detectors). PACTER techniques, which do not require re-calibration for different objects or during long-term operation, allows for longitudinal volumetric imaging of hemodynamics in vasculature a few-millimeters below the skin's surface as discussed in Section IV where in healthy humans, hemodynamic changes in vessels in their feet in response to vascular occlusion have been captured. Single-shot volumetric photoacoustic imaging using a single-element detector may facilitate the early detection and monitoring of peripheral vascular diseases and may find advantageous uses in biometrics and in point-of-care testing.

Certain embodiments pertain to PACTER techniques that can address challenges faced by previous 3D imaging techniques. PACTER techniques provide a highly accessible and efficient solution, paving the way for noninvasive, label-free, and ultrafast volumetric imaging of hemodynamics at depth in humans. With PACTER techniques, a single-element detector encodes information equivalent to that of a large number (e.g., 6400 or larger) of virtual ones, enabling the reconstruction of a tomographic image of vasculature in 3D with just a single laser pulse. PACTER techniques can achieve longitudinal volumetric imaging at a high rate (e.g., 1 kHz to 1 MHz), making it possible to capture fast hemodynamics in the human body in real-time.

Section IV provides demonstrations of PACTER systems showing capability in monitoring vital signs in small animals and visualizing human hemodynamics in response to cuffing, capturing the variability in blood flow speeds. In addition, these demonstrations show that PACTER systems can capture the hemodynamic changes in human foot vessels during vascular occlusion, demonstrating potential as a powerful tool for assessing vascular function in the lower extremities. Enabled by the integrated ultrasonic transducer and the object-independent universal calibration, PACTER systems only need to be calibrated once and are suitable for long-term imaging in unstable environments. PACTER's single-element detector design makes it convenient, affordable, and compact, thus translatable to clinical applications such as home-care monitoring, biometrics, point-of-care testing, and noninvasive hemodynamic monitoring in intensive care units. PACTER's capacity to capture dynamic changes in vascular occlusion presents clinical potential for early detection, enhanced assessment, and more personalized treatment of peripheral vascular diseases. The single-element detector concept in PACTER techniques can also be generalized to other imaging technologies, such as ultrasonography.

### II. PACTER Systems

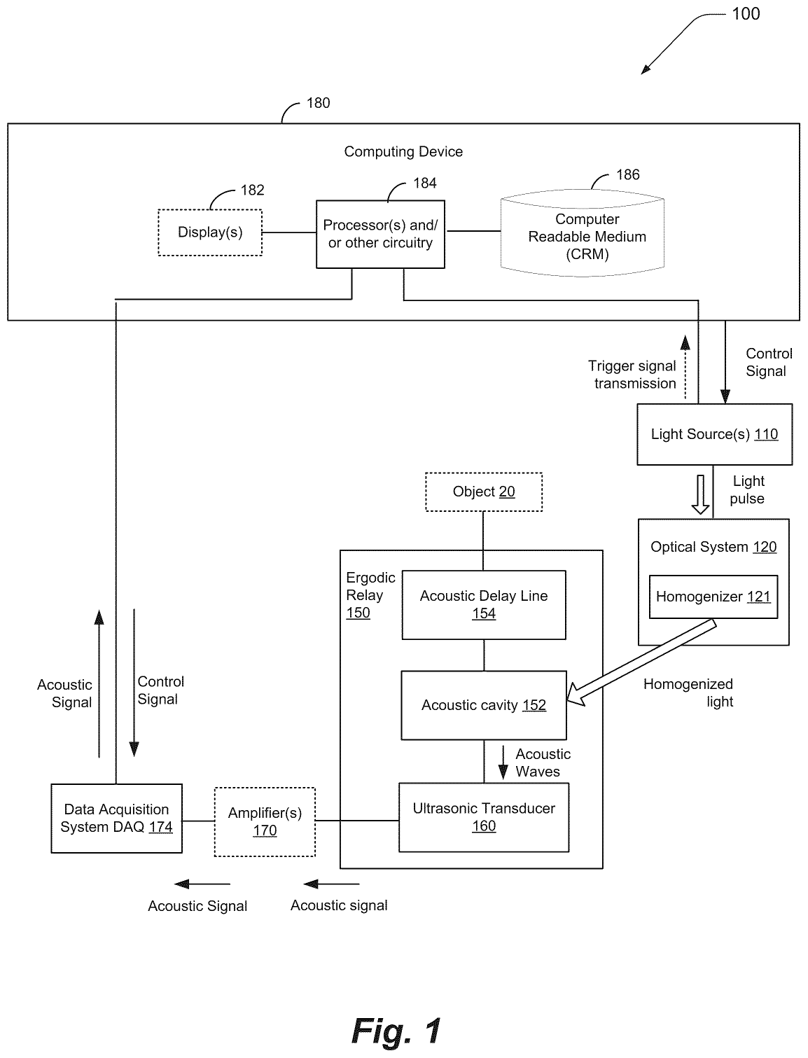

FIG. 1 depicts a block diagram of a PACTER system 100 in an imaging configuration, according to various embodiments. PACTER system 100 includes one or more light sources 110 (e.g., a pulsed laser or a light emitting diode (LED)) that can produce a light pulse for each image acquisition and an optical system 120 in optical communication with light source(s) 110. The optical system 120 includes a homogenizer 121 configured to covert incident light into a widefield, homogenized illumination pattern (homogenized light). In other implementations, the light source(s) may be separate from PACTER system 100.

Returning to FIG. 1, PACTER system 100 also includes an ergodic relay 150 with an acoustic cavity 152 (e.g., a prism), an acoustic delay line 154 (e.g., an optical rod or acoustic waveguide) acoustically coupled to acoustic cavity 152, and an ultrasonic transducer 160 disposed on acoustic cavity 152 to sample photoacoustic waves. In various implementations, ultrasonic transducer 160 is a single-element transducer element disposed on an outer surface of acoustic cavity 152. In some cases, the ultrasonic transducer 160 is an integrated ultrasonic transducer fabricated onto the outer surface of acoustic cavity 152.

The illustrated example is shown during an image acquisition procedure during which an optional (denoted by dashed line) object 20 is present. During this procedure, object 20 is acoustically coupled to acoustic delay line 154 with, for example, water, acoustic gel, etc. The optical system 120 includes a homogenizer 121 configured to homogenize the light pulse from light source(s) 110 and one or more optical elements configured to focus and propagate the homogenized light to acoustic cavity 152. Acoustic cavity 152 is configured to scramble or encode the acoustic waves generated by the photoacoustic effect at the object 20 and acoustic delay line 154 temporally separates the initial and reflected photoacoustic signals.

PACTER system 100 also includes an optional (denoted by dashed line) one or more amplifiers 170 (e.g., ZKL-1R5+ amplifier sold by Mini-Circuits) for boosting one or more photoacoustic signals communicated from ultrasonic transducer 160 and a data acquisition system (DAQ) 174 for digitizing and/or recording the photoacoustic signal(s). Amplifier(s) 170 is in electrical communication with ultrasonic transducer 160 to receive one or more photoacoustic signals and DAQ 174 is in electrical communication with optional amplifier(s) 170 to receive one or more boosted photoacoustic signals.

PACTER system 100 also includes a computing device 180 in electronic communication with DAQ 174 to send control signals and to receive photoacoustic data. Computing device 180 includes one or more processors or other circuitry 184 to perform operations such as image reconstruction, an optional (denoted by dashed line) display 182 in electrical communication with the processor(s) or other circuitry 184, and a computer readable media (CRM) 186 in electronic communication with the processor(s) or other circuitry 184. Computing device 180 is also in electronic communication with light source(s) 110 to send control signals and/or to receive trigger signals. In one aspect, to synchronize recording of photoacoustic signal by DAQ 174 with the light pulse transmission, light source(s) 110 is configured to transmit a trigger signal to computing device 180 that triggers transmission of a control signal to the DAQ 174 to record a photoacoustic signal. In another aspect, the computing device 280 or a separate I/O device sends control signals that trigger the light source(s) 210 to send pulses of light and control the DAQ 274 to simultaneously sample data from the photoacoustic signal received from the photoacoustic element. The electrical communication between system components of the PACTER system 100 may be in wired and/or wireless form.

During an imaging procedure, the object 20 is in contact with the acoustic delay line 154 of the ergodic relay 150 and an acoustic medium such as water or an ultrasound gel is applied between the object 20 and the acoustic delay line 154 to facilitate acoustic coupling. During operation, a pulse of light from the light source(s) 110 is sent through a homogenizer 121 which homogenizes the illumination beam in the imaging volume. The acoustic delay line 154 receives acoustic waves from regions in a field-of-view of object 20 illuminated by the homogenized beam. The acoustic delay line 154 temporally separates the initial photoacoustic signals and the reflected photoacoustic signals. The acoustic cavity 152 further scrambles or encodes acoustic waves received from acoustic delay line 154. The PACTER signals detected by the ultrasonic transducer 160 are amplified by the one or more amplifiers 170 and digitized by DAQ 174. The computing device 180 receives the digitized data and uses the calibrated virtual transducers (photoacoustic signals from calibration pixels of calibration field-of-view) to reconstruct a 3D image for each pulse of light. The computing device 180 may also process the reconstructed image. In some cases, a plurality of pulses of light are triggered over time and a 3D image reconstructed based on each light pulse to generate a sequence of 3D images. The sequence of 3D images can be used to determine a 4D image of the dynamics of the object such as speed of fluid flow.

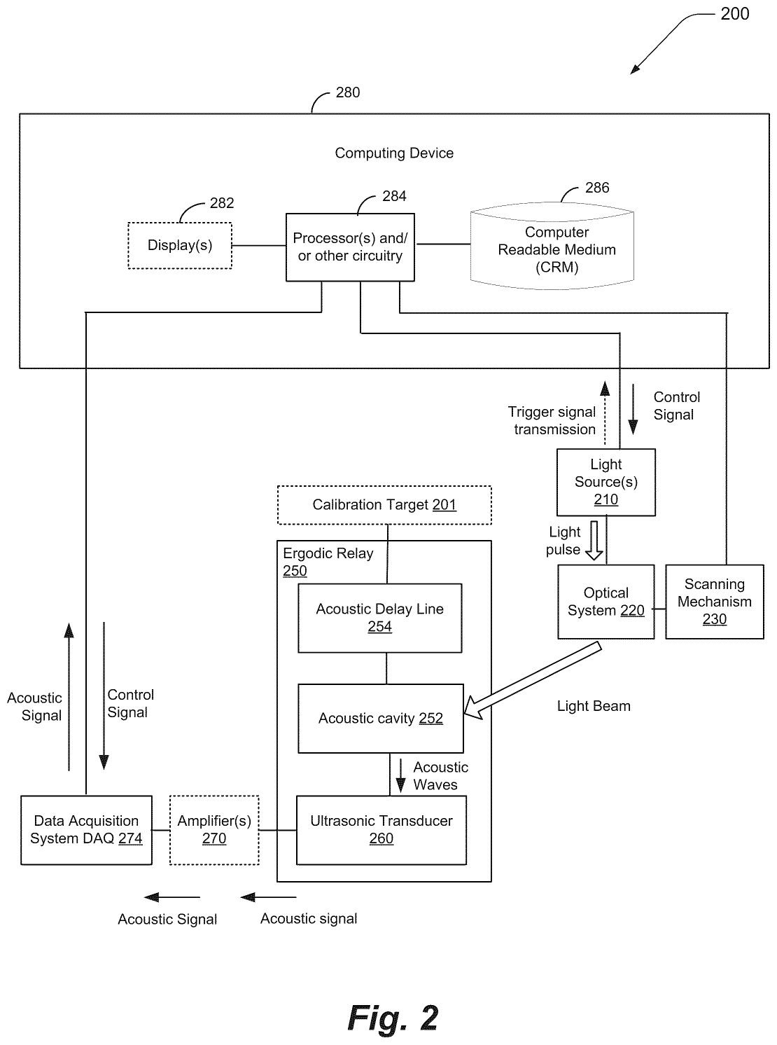

FIG. 2 depicts a block diagram of a PACTER system 200 in a calibration configuration, according to various embodiments. PACTER system 200 includes one or more light sources 210 (e.g., a pulsed laser or a light emitting diode (LED)) that can produce a light pulse for each image acquisition and an optical system 220 in optical communication with light source(s) 210. In other implementations, the light source(s) may be separate from PACTER system 200. PACTER system 200 also includes an ergodic relay 250 with an acoustic cavity 252 (e.g., a prism), an acoustic delay line 254 (e.g., an optical rod or acoustic waveguide) acoustically coupled to acoustic cavity 252, and an ultrasonic transducer 260 disposed on acoustic cavity 252 to sample photoacoustic waves. In various implementations, ultrasonic transducer 260 is a single-element transducer element disposed on an outer surface of acoustic cavity 252. In some cases, the ultrasonic transducer 260 is an integrated ultrasonic transducer fabricated onto the outer surface of acoustic cavity 252.

The illustrated example is shown during a calibration procedure during which an optional (denoted by dashed line) calibration target 201 is present. During the calibration procedure, calibration target 201 is acoustically coupled to acoustic delay line 254 with, for example, water, acoustic gel, etc. PACTER system 200 also includes a scanning mechanism 230 such as one or more translational stages (e.g., PLS-85 translational stage by PI). The optical system 220 is configured to focus and propagate the light beam to acoustic cavity 252. The scanning mechanism 230 is coupled to one or more components of the optical system 220 to steer incident light beam across the field-of-view of the calibration target 201. Acoustic cavity 252 is configured to scramble or encode the acoustic waves generated by the photoacoustic effect at the object 20 and acoustic delay line 254 temporally separates the initial and reflected photoacoustic signals.

PACTER system 200 also includes an optional (denoted by dashed line) one or more amplifiers 270 (e.g., ZKL-1R5+ amplifier sold by Mini-Circuits) for boosting one or more photoacoustic signals communicated from ultrasonic transducer 260 and a data acquisition system (DAQ) 274 for digitizing and/or recording the photoacoustic signal(s). Amplifier(s) 270 is in electrical communication with ultrasonic transducer 260 to receive one or more photoacoustic signals and DAQ 274 is in electrical communication with optional amplifier(s) 270 to receive one or more boosted photoacoustic signals.

PACTER system 200 also includes a computing device 280 in electronic communication with DAQ 274 to send control signals and to receive photoacoustic data. Computing device 280 includes one or more processors or other circuitry 284 to perform operations such as image reconstruction, an optional (denoted by dashed line) display 282 in electrical communication with the processor(s) or other circuitry 284, and a computer readable media (CRM) 286 in electronic communication with the processor(s) or other circuitry 284. Computing device 280 is also in electronic communication with light source(s) 210 to send control signals and/or to receive trigger signals. In one aspect, to synchronize recording of photoacoustic signal by DAQ 274 with the light pulse transmission, light source(s) 210 is configured to transmit a trigger signal to computing device 280 that triggers transmission of a control signal to the DAQ 274 to record a photoacoustic signal. In another aspect, the computing device 280 or a separate I/O device sends control signals that trigger the light source(s) 210 to send pulses of light, control the scanning mechanism 230 to scan the light beam across the calibration field-of-view, and control the DAQ 274 to simultaneously sample data from the photoacoustic signal received from the photoacoustic element. The electrical communication between system components of the PACTER system 200 may be in wired and/or wireless form.

During a calibration procedure, a calibration target 201 is in contact with acoustic delay line 254 of the ergodic relay 250 and an acoustic medium such as water or an ultrasound gel is applied between calibration target 201 and acoustic delay line 254 to facilitate acoustic coupling. During operation, an illumination beam from the light source(s) 210 is steered to illuminate over time different calibration pixel locations in the calibration field-of-view. The acoustic delay line 254 receives acoustic waves from the different pixel locations. The acoustic delay line 254 temporally separates the initial photoacoustic signals and the reflected photoacoustic signals. The acoustic cavity 152 further scrambles or encodes acoustic waves received from acoustic delay line 254. The PACTER signals detected by the ultrasonic transducer 260 are amplified by the one or more amplifiers 270 and digitized by DAQ 274. The computing device 280 uses the photoacoustic signals at the calibration pixel locations as calibrated virtual transducers.

In certain embodiments, a PACTER system requires calibration only once prior to its utilization for imaging. In these cases, the PACTER system does not require calibration for different objects.

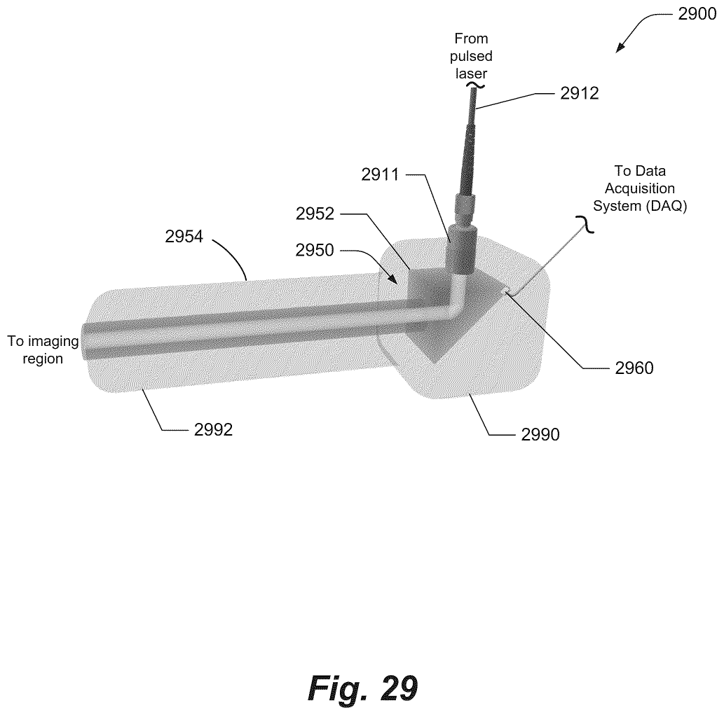

In certain embodiments, a PACTER system includes, or is in communication with, one or more light sources that can provide a pulse of light. Some examples of suitable light sources include pulsed lasers, light emitting diodes, flash lamps, stroboscopes, and pulse-modulated continuous light sources. Any wavelength that can be absorbed by the object being imaged can be used. An example of a suitable light source is a 5-ns pulsed laser beam at 532 nm (e.g., INNOSLAB IS8II-DE pulsed laser with a 1 kHz pulse repetition rate sold by EdgeWave). In implementations using a pulsed laser source, the laser repletion rate employed may be limited by the duration of the photoacoustic signals. In one example, the laser repetition rate is lower than 4 kHz. In hemodynamic implementations, a high speed pulse laser (e.g., Q-switched laser with a variable repetition rate from 0 to 3.5 MHz such as Spectra-Physics Quasar GR95) may be used to be able to rapidly capture multiple 3D images over time. In implementations using a laser source, the laser source may include an iris to adjust the beam diameter. In some cases, a PACTER system includes the one or more light sources. In other cases, the one or more light sources may be a separate component. For example, a PACTER system may include a multi-mode fiber (e.g., multi-mode fiber 2912 in FIG. 29) in optical communication with the one or more light sources to deliver the excitation light to the ergodic relay.

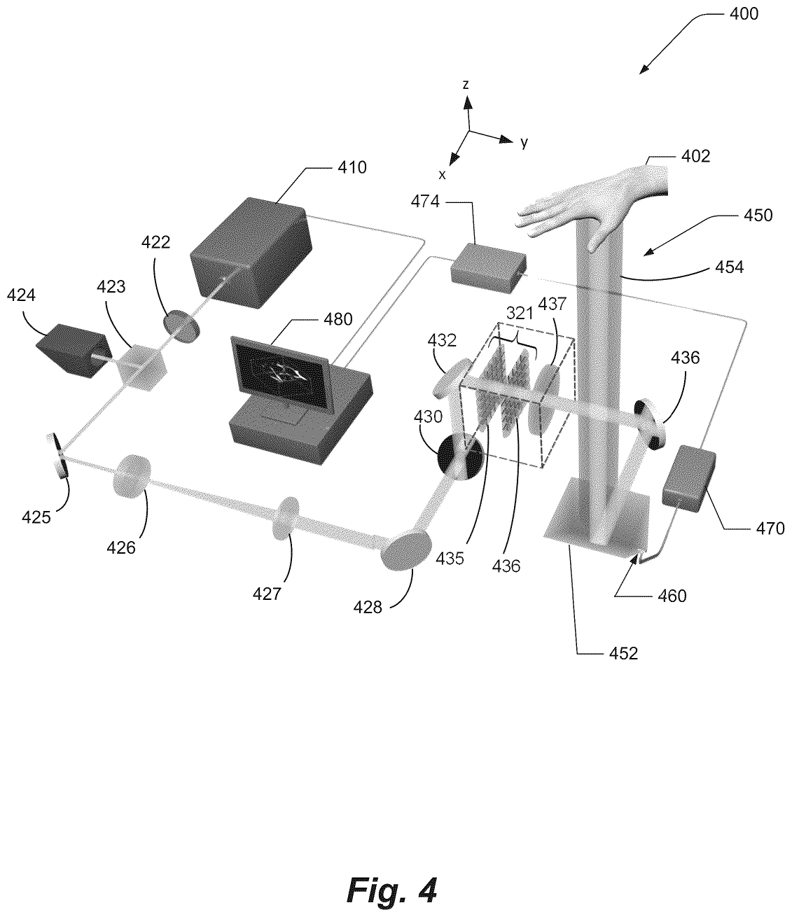

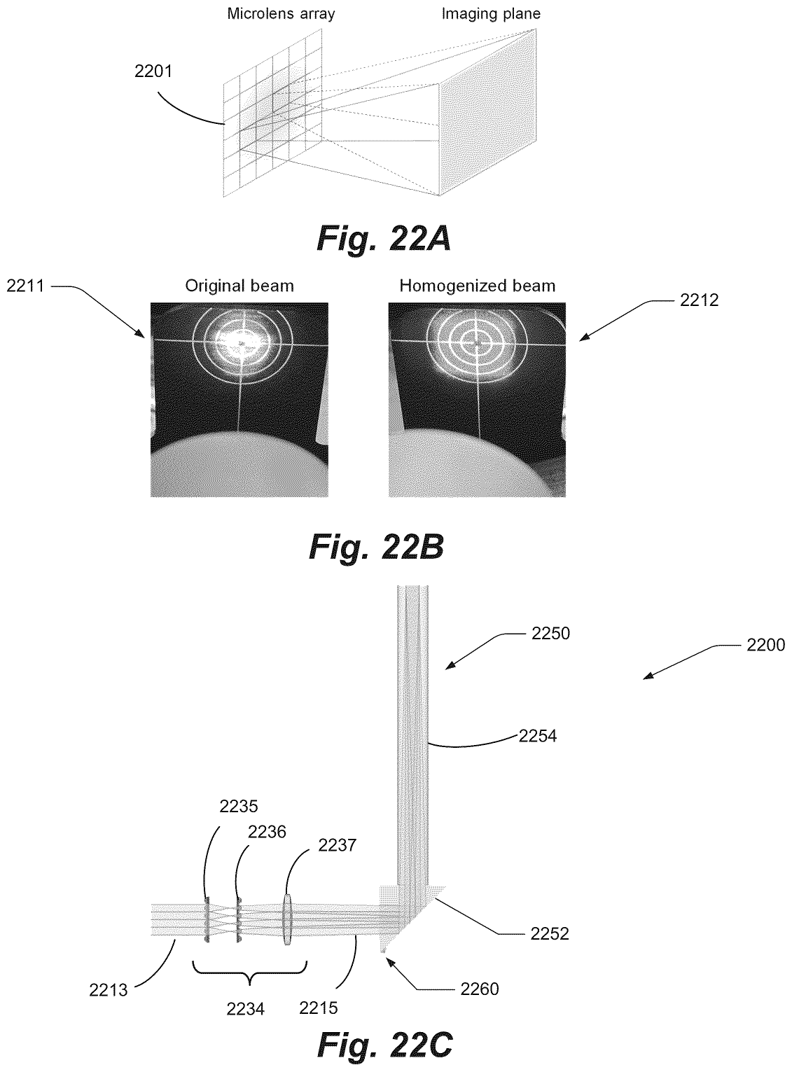

In imaging configurations, widefield homogenized light is provided to the acoustic cavity of the ergodic relay during operation. In these implementations, the optical system may include a homogenizer (e.g., homogenizer 121 in FIG. 1, homogenizer 321 in FIG. 4, or fly's eye homogenizer 2234 in FIG. 22C) that can covert incident light into a widefield, homogenized illumination pattern. In some cases, the homogenized illumination pattern may have the same shape and width as that of the field-of-view (FOV) used during calibration as discussed in Section V(A). In other cases, the homogenized illumination pattern is larger or smaller than the calibration FOV. In addition, or alternatively, the one or more light sources may provide homogenized light (uniform light). In one aspect, the homogenizer may include one or more microlens arrays (e.g., first microlens array 435 and second microlens array 436 in FIG. 4 or first microlens array 2235 and second microlens array 2236 in FIG. 22C). An example of a suitable microlens array is #64-480 lens array sold by Edmund Optics. The microlens arrays may be rectangular arrays, circular arrays, linear arrays, or any combination thereof. An example of a suitable homogenizer is a fly's eye homogenizer (e.g., fly's eye homogenizer 2234 in FIG. 22C) described in Section V. In other implementations, another homogenizer may be used such as, e.g., diffusers, integrating rods, diffractive optical elements. In calibration implementations, a PACTER system may omit the homogenizer.

A PACTER system includes an ergodic relay (ER) having an acoustic cavity (e.g., a silica prism) configured to scramble or encode acoustic waves and an acoustic delay line (e.g., an optical rod or an acoustic waveguide) acoustic coupled to the acoustic cavity. The acoustic delay line is configured to temporally separate the initial photoacoustic signals and the reflected photoacoustic signals. In one example, an ergodic relay includes a silica optical rod fused to a silica prism. The ergodic relay can be used as an encoder to transform photoacoustic signals from acoustic waves at different input positions into unique temporal signals. The ergodic relay not only introduces a characteristic delay between the delivery of the illumination beam and the reception of the photoacoustic signal by the ultrasonic transducer element but also stretches and scrambles the signal in time, encoding the spatial location of the photoacoustic signal's source in a complex temporal pattern.

The acoustic cavity is generally configured to scramble or encode acoustic waves, e.g., generated by the photoacoustic effect (photoacoustic waves) at the object being imaged. The acoustic cavity may be of various shapes and dimensions that can scramble acoustic waves. An example of an acoustic cavity is a prism such as a right-angle prism. Other suitable acoustic cavities include glass/quartz plates (e.g., slides, coverslips), silicon wafers, etc. An example of a suitable prism is the PS611 prism sold Thorlabs, Inc. of Newton, New Jersey with a 25 mm right-angle edge length. Other suitable prisms are the PS612 prism, the PS608 prism, the PS613 prism, etc. sold Thorlabs, Inc. of Newton, New Jersey. The acoustic cavity may be fabricated of one or more transparent materials. Some examples of suitable transparent materials include glass, polymethyl methacrylate, epoxy, and polycarbonate.

The acoustic delay line is generally configured to temporally separate the initial photoacoustic signals and the reflected photoacoustic signals. Some examples of suitable acoustic delay lines are an optical rod and an acoustic waveguide. The acoustic delay line may be made of various materials and have different dimensions. In one example, the acoustic delay line has a length between 50 mm and 300 mm. In another aspect, the acoustic delay line has a length in a range of 5 mm and 30 mm. In one example, the acoustic delay line has a circular cross section with a diameter in range between 10 mm and 50 mm. An example of a suitable optical rod is the optical rod sold by VY Optoelectronics having an 18 mm diameter, 175 mm length, top and bottom surfaces polished to 60-40 surface quality. The acoustic delay line may be fused directly to the acoustic cavity or coupled via an adhesive material (e.g., polyester resin) to the acoustic cavity. The acoustic delay line may have an object surface configured to contact an object being imaged during operation.

In certain embodiments, the ergodic relay includes one or more acoustic impedance-matching layers between the object being imaged and the other portion of the ergodic relay. For example, the one or more acoustic impedance-matching layers may be located at a surface of the ergodic delay that is designed to contact the object being imaged. An example of a suitable acoustic impedance-matching layer is a quarter-wavelength impedance-matching layer (Z_{match}=\sqrt{Z_{object}Z_{ER}}) where Z_{object} and Z_{ER} are the acoustic impedances of the object and the ergodic relay, respectively. Another example of a suitable acoustic impedance-matching layer is a cascaded impedance-matching layer. Another example of a suitable acoustic impedance-matching layer is a gradient impedance-matching layer.

In some embodiments, an ergodic relay includes a prism and an optical rod (e.g., 18 mm diameter, 175 mm length, top and bottom surfaces polished to 60-40 surface quality). The prism may be a right-angle prism such as, e.g., PS611 prism having 25 mm right-angle edge length sold by Thorlabs. In some cases, the prism and the optical rod are both made of ultraviolet (UV) fused silica, which has good optical transparency and low acoustic attenuation. In some cases, one or more edges of the prism are ground by a saw such as a diamond saw (e.g., SYJ-150 saw sold by MTI Co.) following a sawtooth pattern to obtain chaotic boundaries. The prism and the optical rod may be glued by UV-curing optical adhesive (e.g., NOA68 adhesive sold by Norland Products), following exposure under UV light for 12 hr.

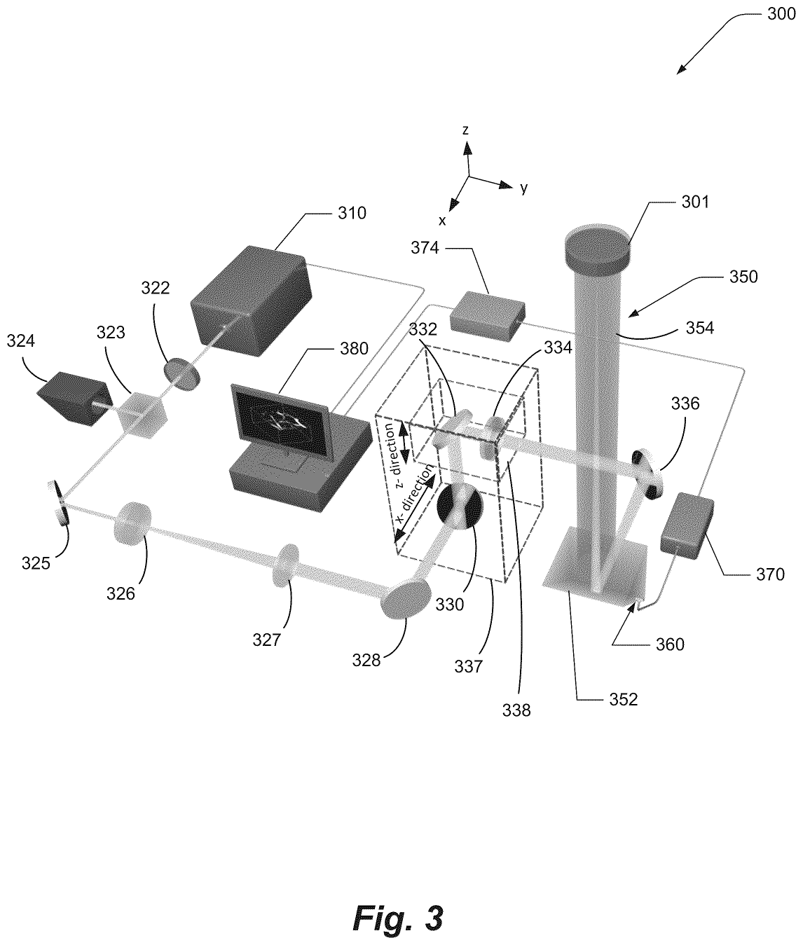

In calibration implementations, a PACTER system may also include a scanning mechanism (e.g., one or more translational stages) coupled to one or more components of the optical system to be able to steer the illumination light beam to different locations along a field-of-view of the calibration target (calibration FOV). An example of a suitable translation stage is a PLS-85 translational stage by PI. Each translation stage may be controller by a motor driver such as, e.g., CW215 motor driver sold by Circuit Specialists. According to one aspect, the scanning mechanism includes a first motorized stage and a second motorized stage (e.g., first motorized stage 337 and second motorized stage 338 in FIG. 3) to translate the components along two orthogonal directions. In this aspect, two motor drivers may control the movement.

In certain embodiments, a PACTER system includes a data acquisition system (DAQ) for digitizing and/or recording photo acoustic signals. In one aspect, the DAQ is a single channel DAQ that provide one-to-one mapped association to a single element ultrasonic transducer. Although the PACTER systems of certain implementations include a DAQ (e.g., a DAQ card) for data acquisition, other implementations may include other devices such as one or more microcontrollers. An example of a suitable DAQ is ATS9350 data acquisition card sold by AlazarTech. In some cases, the DAQ may be installed on the computing device.

Optionally, a PACTER system includes one or more amplifiers (e.g., amplifier(s) 170 in FIG. 1) that boost the signal detected by the ultrasonic transducer. The one or more amplifiers may provide one-to-one mapped association with a single-element ultrasonic transducer. The one dedicated amplifier channel is configured to amplify photoacoustic signals detected by the single-element ultrasonic transducer. In various embodiments, a PACTER system includes two low-noise amplifiers (e.g., ZKL-1R5+ sold by Mini-Circuits).

In certain embodiments, a PACTER system includes a computing device having one or more processors or other circuitry, an optional display in electrical communication with the processor(s), and a computer readable media (CRM) in electronic communication with the processor(s) or other circuitry. The computing device may include an input/output (I/O) device (e.g., multifunctional input/output (I/O)) for controlling the one or more light sources, the scanning mechanism, and/or the DAQ.

The computer readable media (CRM) may be, e.g., a non-transitory computer readable media. The computing device 180 is in electronic communication with the light source(s) to send control signals to trigger the illumination (e.g., triggering laser pulses). The computing device is in electrical communication with the one or more DAQs to receive data transmissions and/or to send control signal(s). The computing device 180 may also be in electronic communication with the one or more pre-amplifiers to send control signal(s), e.g., to adjust amplification. The electrical communication between system components of the PACTER system may be in wired and/or wireless form. One or more of the electrical communications between components of the PACTER system may be able to provide power in addition to communicate signals. The computing device may be, for example, a personal computer, an embedded computer, a single board computer (e.g., Raspberry Pi or similar), a portable computation device (e.g., tablet), a controller, or any other computation device or system of devices capable of performing the functions described herein. The computing device may also be in electronic communication with a scanning mechanism to send control signals to control the movement and/or hold positions of the one or more optical components. The processor(s) are in electrical communication with the CRM to store and/or retrieve data such as the photoacoustic signal data. The one or more processor(s) and/or other circuitry are in electrical communication with the optional display to display data. The computing device may also include a user input component for receiving data from a user. In one embodiment, a computing device includes a GPU.

The one or more processors and/or other circuitry may execute instructions stored on the CRM to perform one or more operations of PACTER methods. In certain implementations, the processor(s) and/or other circuitry execute instructions to perform one or more of: 1) reconstruction of 3D PACTER images and 2) generation of 4D image data. For example, the processor(s) and/or other circuitry and/or one or more external processors may execute instructions that communicate control signals to a scanning mechanism to scan the light beam across field-of-view of a calibration target and send control signals to the DAQ(s) to simultaneously record photoacoustic signals detected by the ultrasonic transducer element.

In one embodiment, a PACTER system includes a multifunctional input/output (I/O) device (e.g., PCIe-6321 I/O device sold by National Instruments) configured to execute instructions to (1) trigger the one or more light sources such a pulsed laser, (2) drive the scanning device (e.g., motorized stages) during calibration, and/or (3) acquire the data by the DAQ. In one example with one or more motorized stages and a pulsed laser, the PACTER signals were acquired at a sampling rate of 250 megasamples per second, and a sampling length of 65,532 data points per acquisition. In this example, due to the distance between the object and the ultrasonic transducer, a 28-μs delay was added to the data acquisition following the laser trigger. During calibration, to improve the signal-to-noise ratio (SNR) of the signal, the acquisition was repeated 500 times at each calibrated virtual transducer and the averaged signal used for PACTER reconstruction. To prevent motor backlash, the data was acquired only when the motor was moving forward; the acquisition stopped when the motor returned. During imaging, to improve the temporal resolution of the system, no signal averaging was used, and the motor scanning was disabled.

In some implementations, the PACTER system includes one or more communication interfaces (e.g., a universal serial bus (USB) interface). Communication interfaces can be used, for example, to connect various peripherals and input/output (I/O) devices such as a wired keyboard or mouse or to connect a dongle for use in wirelessly connecting various wireless-enabled peripherals. Such additional interfaces also can include serial interfaces such as, for example, an interface to connect to a ribbon cable. It should also be appreciated that the various system components can be electrically coupled to communicate with various components over one or more of a variety of suitable interfaces and cables such as, for example, USB interfaces and cables, ribbon cables, Ethernet cables, among other suitable interfaces and cables.

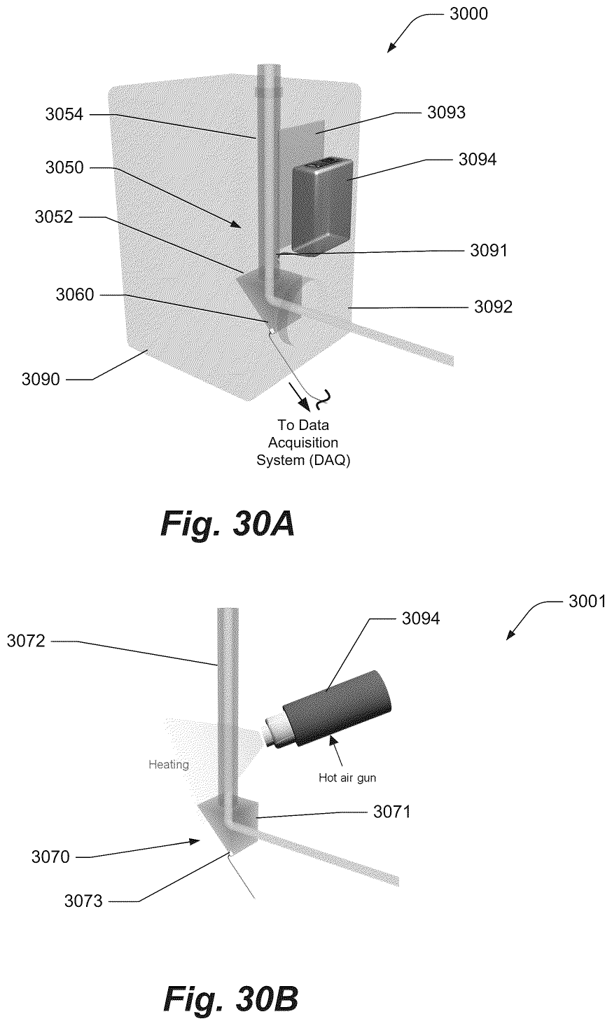

In certain embodiments, the PACTER system may include a temperature stabilizing system (e.g., temperature stabilizing box 3990 in FIG. 30A) to stabilize temperature fluctuations during operation to try to avoid change of speed of sound due to temperature fluctuations. The temperature stabilizing system may include one or more thermal insulating layers and/or temperature stabilizing elements. In one example, the temperature stabilizing system includes a temperature stabilizing box (e.g., temperature stabilizing box 3090 in FIG. 39A). In one case, the entire ergodic relay is sealed within the temperature stabilizing box.

In one embodiment, the temperature stabilizing system includes a temperature stabilizing box (e.g., temperature stabilizing device 3090 in FIG. 30A) regulated by a thermocouple (e.g., SA1-E thermocouple sold by Omega Engineering), a heating pad (e.g., SRFG-303/10 heating pad sold by Omega Engineering), and a temperature controller (e.g., Dwyer 32B-33 temperature controller sold by Cole-Parmer). FIG. 30A depicts a PACTER system 3000 including a temperature stabilizing system with a temperature stabilizing device 3000 regulated by a thermocouple 3091, a heating element 3093, and a temperature controller 3094.

FIG. 3 depicts a schematic diagram of a PACTER system 300 in a calibration configuration, according to an embodiment. PACTER system 300 includes a light source in the form of a pulsed laser 310 (e.g., a 5-ns pulsed laser beam at 532 nm such as, e.g., INNOSLAB IS8II-DE pulsed laser with a kHz pulse repetition rate sold by EdgeWave), an optical system, an ergodic relay 350, one or more low-noise amplifiers 370 (e.g., ZKL-1R5+ amplifier sold by Mini-Circuits), a low-pass filter (not shown) (e.g., BLP-70+ low pass filter sold by Mini-Circuits), a data acquisition system (DAQ) 374 (e.g., ATS9350 data acquisition card sold by AlazarTech), and a computing device 380 in electronic communication with pulsed laser 310 and DAQ 374. Ergodic relay 350 includes an acoustic cavity in the form of a right-angle silica prism 352 that is fused to an acoustic delay line in the form of a silica optical rod 354. Ergodic relay 350 also includes an integrated ultrasonic transducer 360 fabricated onto an outer surface of right-angle silica prism 352. The optical system includes a half-wave plate 322 (e.g., WPH10M-532 half-wave plate sold by Thorlabs), a polarizing beam splitter 323 (e.g., PBS25-532-HP beam splitter sold by Thorlabs), a beam trap 324 (e.g., BT610 beam trap sold by Thorlabs), a first mirror 325, a first lens 326, a second lens 327, a second mirror 328, a third mirror 330, a fourth mirror 332, a third lens 334, and a sixth mirror 336. First lens 326 and second lens 327 (e.g., ACN254-050-A and AC254-100-A lenses sold by Thorlabs) expand the beam reflected by first mirror 325. Low-noise amplifier(s) 370 is in electronic communication with integrated ultrasonic transducer 360 and DAQ 374 (e.g., ATS9350 data acquisition card sold by AlazarTech). Modifications, additions, or omissions may be made to PACTER system 300 as shown in FIG. 3 without departing from the scope of the disclosure. For example, in other implementations PACTER system 300 may omit one or more mirrors.

The illustrated example is shown at an instant during a calibration procedure during which a calibration target 301, which is generally a uniform optical absorber, is placed in acoustic communication (e.g., via acoustic gel or water) with a distal end of silica right-angle silica prism 352. PACTER system 300 also has a scanning mechanism that includes a first motorized stage 337 (x-direction) (e.g., PLS-85 translational stage by PI) and a second motorized stage 338 (z-direction) (e.g., PLS-85 translational stage by PI). The third mirror 330, fourth mirror 332, and third lens 334 are mounted to first motorized stage 337 (x-direction) and a second motorized stage 338 (z-direction) to steer the focused laser beam across a calibration field-of-view (FOV) in an x-y plane to a plurality of scanning locations at calibration target 301 during a calibration procedure. The illustration shows the optical path of the light beam through components of PACTER system 300.

An acoustic medium such as water or an ultrasound gel (e.g., Aquasonic 100 ultrasound gel sold by ParkerLabs) may be applied between the calibration target 301 and a surface of a distal end of ergodic relay 350 to facilitate acoustic coupling. In PACTER system 300, the power of the laser beam from pulsed laser 310 is controlled by the half-wave plate 322 and polarizing beam splitter 323. During operation, the beam reflected by the polarizing beam splitter 323 is sent to beam trap 324. The beam transmitted through the polarizing beam splitter 323 is expanded by a beam expander consisting of first lens 326 and second lens 327. During the calibration procedure, the expanded beam is steered across the calibration FOV by the third mirror 330, fourth mirror 332, and third lens 334 mounted to the first motorized stage 337 (x-direction) and a second motorized stage 338 (z-direction). An iris (not shown) is used to adjust the beam diameter (e.g., to be about 2 mm) and the beam is sent through lens 334 and focused at, or near, a distal end of the ergodic relay 350. The PACTER signals detected by the ultrasonic transducer 360 were amplified by the one or more low-noise amplifiers 370, filtered by the low-pass filter (not shown), and digitized by DAQ (e.g., ATS9350 data acquisition card sold by AlazarTech). The DAQ 374 may be installed on the computing device 350. In one embodiment, the PACTER system 300 includes a multifunctional input/output (I/O) device (e.g., PCIe-6321 I/O device sold by National Instruments) to control the one or more light sources 310, the first motorized stage 337, the second motorized stage 338, and/or the DAQ 374.

The calibration target 301 is typically a uniform optical absorber such as lysed bovine blood. In one implementation, the calibration target 301 includes a container with a window at its bottom sealed with an optically and ultrasonically transparent disposable polyethylene membrane. The container is filled with bovine blood, which is used as a uniform optical absorber for calibration. Lysed bovine blood may be useful as a calibration target particularly where primary imaging objects are blood vessels. Using first motorized stage 337 and second motorized stage 338, the positions of the pair of mirrors 332 and 334 is controlled to steer the focused laser beam across the field-of-view (FOV) in an x-y plane and the PACTER signals are recorded by DAQ 374 at each of a plurality of scanning positions.

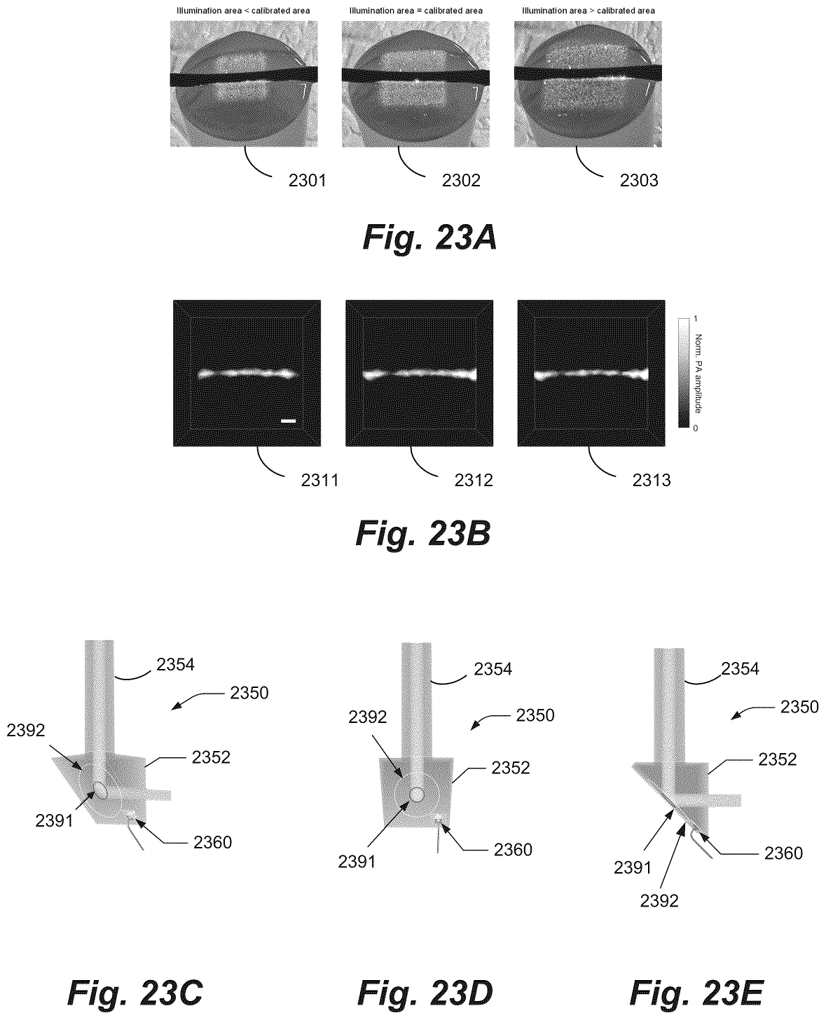

After calibration, the calibration target can be removed from the PACTER system to ready the system for imaging once a homogenizer such as a fly's eye homogenizer is introduced. Section V(A) describes an example of a fly's eye homogenizer. The homogenizer converts the incident laser beam into a widefield, homogenized illumination pattern. In some cases, the homogenized illumination pattern has the same shape and width as that of the calibration field-of-view (FOV) as discussed in Section V(A). In other cases, the illumination pattern is larger or smaller than the calibration FOV. FIG. 23A depicts photographs 2301, 2302, 2303 of a PACTER imaging setup of a black wire when the illumination area is smaller (left), equivalent (middle), and larger (right) compared with the calibrated area. FIG. 23B depicts 3D PACTER images 2311, 2312, 2313 of the black wire in FIG. 23A acquired when the illumination area is smaller (left), equivalent (middle), and larger (right) compared with the calibrated area. Scale bar is 1 mm. As shown, the illumination area/pattern can be smaller or larger than the calibrated FOV. The scanning mechanism (e.g., first and second motorized stages 337 and 338 in FIG. 3) may also be removed from the PACTER system for an imaging procedure.

FIG. 4 depicts a schematic diagram of PACTER system 400 in an imaging configuration, according to an embodiment. PACTER system 400 includes a light source in the form of a pulsed laser 410 (e.g., a 5-ns pulsed laser beam at 532 nm such as, e.g., IS8II-DE pulsed laser with a kHz pulse repetition rate sold by EdgeWave), an optical system, an ergodic relay 450, one or more low noise amplifiers 470 (e.g., ZKL-1R5+ amplifier sold by Mini-Circuits), a low-pass filter (not shown) (e.g., BLP-70+ low pass filter sold by Mini-Circuits), a data acquisition system (DAQ) 474 (e.g., ATS9350 data acquisition card sold by AlazarTech), and a computing device 480 in electronic communication with pulsed laser 410 and DAQ 474. Ergodic relay 450 includes an acoustic cavity in the form of a right-angle silica prism 452 that is fused to an acoustic delay line in the form of a silica optical rod 454. Ergodic relay 450 also includes an integrated ultrasonic transducer 460 fabricated onto an outer surface of right-angle silica prism 452. The optical system includes a half-wave plate 422 (e.g., WPH10M-532 half-wave plate sold by Thorlabs), a polarizing beam splitter 423 (e.g., PBS25-532-HP beam splitter sold by Thorlabs), a beam trap 424 (e.g., BT610 beam trap sold by Thorlabs), a first mirror 425, a first lens 426, a second lens 427, a second mirror 428, third mirror 430, a fourth mirror 432, a homogenizer 421 including a first microlens array 435 (e.g., #64-480 lens array sold by Edmund Optics) and a second microlens array 436 (e.g., #64-480 lens array sold by Edmund Optics), a spherical lens 437 (e.g., AC254-250-A lens sold by Thorlabs), and a fifth mirror 436. First lens 426 and second lens 427 (e.g., 1254-050-A and AC254-100-A lenses sold by Thorlabs) expand the light beam reflected by first mirror 425. Low-noise amplifier(s) 470 is in electronic communication with integrated ultrasonic transducer 460 and DAQ 474 (e.g., ATS9350 data acquisition card sold by AlazarTech). First microlens array 435 and second microlens array 437 convert the incident light beam into a widefield, homogenized illumination pattern (homogenized light) and spherical lens 437 (e.g., AC254-250-A lens sold by Thorlabs) focuses the homogenized light to provide a light beam of focused homogenized light. The illustrated example is shown at an instant during an imaging procedure during which an object 402 is placed in acoustic communication (e.g., via acoustic gel or water) with a distal end of silica prism 452. The illustration shows the optical path of the light beam through components of PACTER system 400 during the imaging procedure. Modifications, additions, or omissions may be made to PACTER system 400 as shown in FIG. 4 without departing from the scope of the disclosure. For example, in other implementations PACTER system 400 may omit one or more mirrors.

During the imaging procedure, to acquire imaging data, the object 402 is placed directly in contact the optical rod 454 and an acoustic medium such as water or an ultrasound gel (e.g., Aquasonic 100, ParkerLabs) is applied between the object 402 and a surface of a distal end of ergodic relay 450 to facilitate acoustic coupling. In PACTER system 400, the power of the laser beam from pulsed laser 410 is controlled by the half-wave plate 422 and polarizing beam splitter 423. During operation, the beam reflected by the polarizing beam splitter 423 is sent to beam trap 424. The beam transmitted through the polarizing beam splitter 423 is expanded by a beam expander consisting of first lens 426 and second lens 427. An iris (not shown) is used to adjust the beam diameter (e.g., to be about 6 mm) and the beam is sent through a homogenizer including first microlens array 435, second microlens array 436, and lens 437 (e.g., AC254-250-A lens sold by Thorlabs), which homogenized the beam in the imaging volume. The PACTER signals detected by the ultrasonic transducer 460 are amplified by the one or more low-noise amplifiers 470, filtered by the low-pass filter (not shown), and digitized by DAQ (e.g., ATS9350 data acquisition card sold by AlazarTech) 374. The DAQ 474 may be installed on the computing device 450. In one embodiment, the PACTER system includes a multifunctional input/output (I/O) device (e.g., PCIe-6321 I/O device sold by National Instruments) to control the light source 410, and the DAQ 474.

A PACTER signal is recorded by DAQ 474 from acoustic waves generated by the object 402 and detected by integrated ultrasonic transducer 460 following a laser light pulse. The DAQ 474 digitizes the photoacoustic signal. The computing device 480 uses the calibrating photoacoustic signals from a plurality of calibrating pixels generated from a calibration process as a plurality of virtual detectors to reconstruct a 3D image of the object 402.

In certain embodiments, an ergodic relay of a PACTER system includes an integrated single-element ultrasonic transducer, e.g., that is fabricated (e.g., sputtered) directly onto an outer surface of an acoustic cavity. The single-element ultrasonic transducer may be based on a lead magnesium niobate-lead titanate (PMN-PT) single crystal, for example. The single-element ultrasonic transducer may be fabricated directly onto the surface of the acoustic cavity in a region where the single-element ultrasonic transducer does not interact with the illumination laser beam. Fabricating the ultrasonic transducer directly onto the surface may enhance the detection sensitivity and improve the stability of the PACTER system for long-term imaging in unstable environments. In other embodiments, a single-element ultrasonic transducer element may be a separate pre-fabricated component that is coupled to the acoustic cavity with, e.g., a resin.

An example of a suitable single-element ultrasonic transducer element is a pin-shaped ultrasound transducer. A commercially-available example of a single-element ultrasonic transducer element is a VP-0.5 transducer made by CTS Electronics, Inc., which has a 10 MHz central frequency and 0.5 mm element size. Another commercially-available example is the VP-0.5−20 MHz transducer made by CTS Electronics, Inc., which has a 20 MHz central frequency, 56% one-way bandwidth, and 0.5 mm element size.

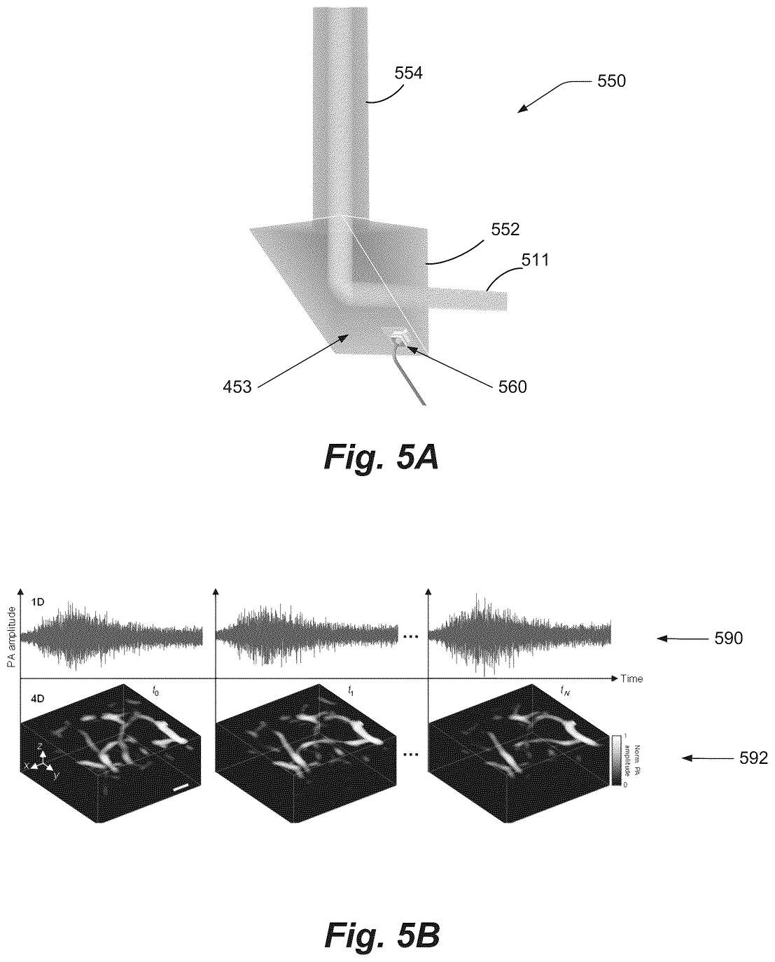

FIG. 5A depicts a schematic diagram of an ergodic relay 550, according to an embodiment. The ergodic relay 550 includes an acoustic cavity in the form of a right-angle glass prism 550, a silica optical rod 554 fused to prism 552, and an integrated single-element ultrasonic transducer 560 fabricated onto a hypotenuse outer surface 553 of right-angle prism 552 in an area where the transducer does not interact with the illumination laser beam 511. In other implementations, integrated single-element ultrasonic transducer 560 may be fabricated on another surface of prism 552 in another area that does not interfere with the illumination beam 511.

FIG. 23C depicts a schematic drawing of a view an ergodic relay 2350 with an integrated single-element ultrasonic transducer 2360, according to an embodiment. Ergodic relay 2350 also includes a prism 2352 fused to a glass rod 2354. The illustration shows an excitation beam area 2391. In this example, the single-element ultrasonic transducer 2360 is fabricated onto the hypotenuse surface of the prism 2352 in a region outside a clear area 2392 where the illumination laser beam does not interact with the single-element ultrasonic transducer 2360. FIG. 23D depicts a schematic drawing of another view of ergodic relay 2350 in FIG. 23C. FIG. 23E depicts a schematic drawing of another view of ergodic relay 2350 in FIG. 23C.

In some examples, the single-element ultrasonic transducer is based on a lead magnesium niobate-lead titanate (PMN-PT) single crystal, which may achieve exceptional piezoelectric performance, such as high piezoelectric constant (d₃₃) and electromechanical coupling coefficient (kₜ).

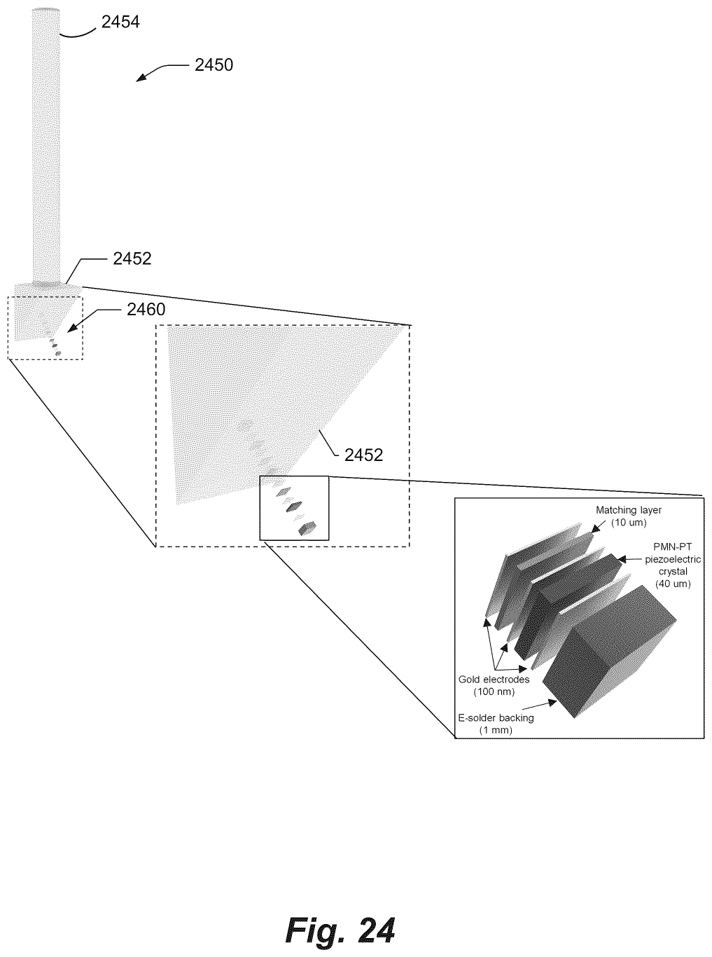

FIG. 24 is a schematic representation of sputter fabrication of material layers of an integrated single-element ultrasonic transducer 2460 onto a hypotenuse surface of a prism 2452 of an ergodic relay 2450, according to an embodiment. In this example, integrated single-element ultrasonic transducer 2460 is based on a lead magnesium niobate-lead titanate (PMN-PT) single crystal (also referred to as an “integrated PMN-PT transducer”). The material layers of integrated PMN-PT transducer 2460 are sputter deposited onto the hypotenuse surface of prism 2452. The illustration depicts a zoomed-in view of the dashed line box and a zoomed-in view of the solid line box. As shown, one of the gold electrodes of integrated PMN-PT transducer 2460 may be sputter deposited onto the hypotenuse surface of prism 2452 of ergodic relay 2450. Because one of the gold electrodes of the integrated PMN-PT transducer 2460 is directly sputtered onto the surface of the ergodic relay, the PACTER signals inside the ergodic relay 2450 may reach the integrated ultrasonic transducer with maximum transmission. The integrated PMN-PT transducer 2460 and the ergodic relay are an integrated piece that may facilitate stable data acquisition for long-term imaging (e.g., imaging in vivo) in unstable environments.

FIG. 5B depicts graphs 590 of 1D(t) PACTER signals detected by single-element ultrasonic transducer 560 in FIG. 5A at time instances t₀, t₁, . . . t_{N} and a 4D (xyzt) reconstructed image 592 at time instances t₀, t₁, . . . ty of human palmar vessels from the 1D(t) PACTER signals, according to embodiments.

A single-element ultrasonic transducer generally acquires a 1D signal in the time domain as shown in the three graphs 590 of FIG. 5B. However, when used in conjunction with an ergodic relay having an acoustic delay line, a PACTER system can encode spatiotemporal information equivalent to those captured by 6,400 detectors, which can then be used to reconstruct a 3D map of the optical absorbers in the imaging volume. With a kilohertz laser repetition rate, PACTER reconstruction can use the 1D (t) signals to generate a thousand 3D (xyz) volumes per second, leading to a high-speed 4D (xyzt) image of optical absorption in, e.g., human palmar vessels, as shown in the reconstructions 592 in FIG. 5B.

### III. Methods

(A) Method of Fabricating Ergodic Relay with Integrated Single-Element Ultrasonic Transducer

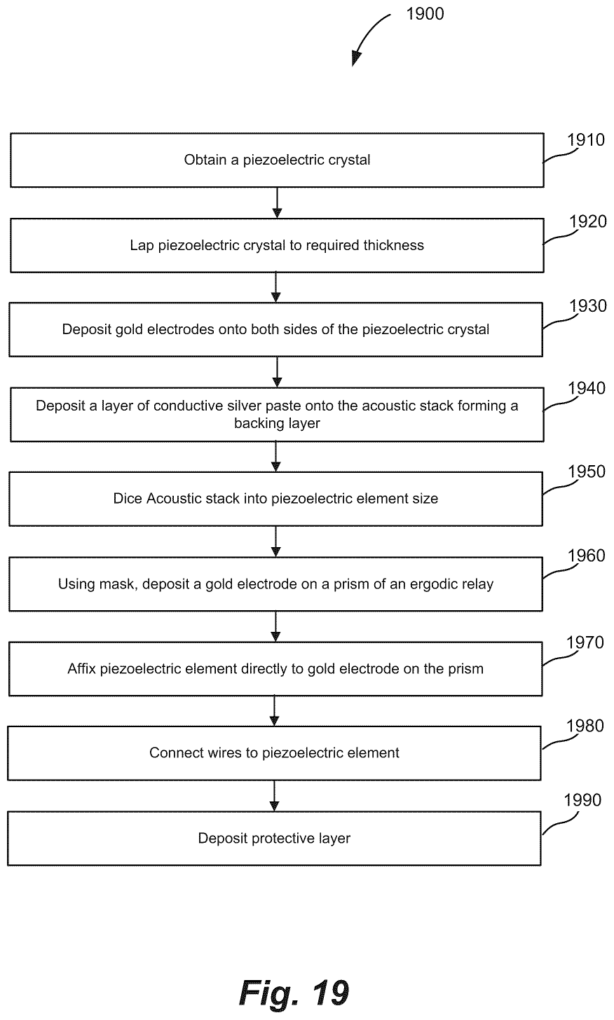

FIG. 19 is a flowchart depicting operations of a method of fabricating an ergodic relay with an integrated single-element ultrasonic transducer. FIG. 24 is a schematic drawing depicting the integrated single-element ultrasonic transducer fabricated on the ergodic relay.

At operation 1910, a piezoelectric crystal is obtained. In some cases, a PMN-PT piezoelectric single crystal (e.g., PMN-PT piezoelectric single crystal sold by CTS Corporation) is the core component for acoustic-electrical conversion due to the excellent piezoelectric coefficient and high permittivity, which is suitable for high-frequency transducers with small aperture sizes because of the general electrical impedance matching (e.g., 50 ohms). Based on the material parameters, a transducer modeling software (e.g., PiezoCAD) based on Krimboltz, Leedom, and Mattaei (KLM) equivalent circuit model was employed to simulate and optimize the design of the transducer. In one example, 30 MHz PMN-PT transducer with a small active aperture size of 0.4× 0.4 mm² was determined and obtained. The piezoelectric element has a central frequency of 30 MHz.

At operation 1920, the piezoelectric crystal is lapped to the required thickness (e.g., 40-μm). At operation 1930, gold electrodes are deposited (e.g., sputter deposited) onto both sides of the piezoelectric crystal. Some examples of a thickness of the gold electrodes include 100 nm, 150 nm, etc.

At operation 1940, a layer of conductive silver paste (e.g., E-solder 3022) is deposited onto the acoustic stack as a backing layer.

At operation 1950, the acoustic stack is diced into designed piezoelectric element size. Some examples of a designed piezoelectric element size include 0.4×0.4 mm².

At operation 1960, using Kapton tape or other tape as a mask, a gold electrode is deposited (e.g., sputtered) on a region (e.g., a corner) of a surface of a prism of an ergodic relay. The prism may be fused to an optical rod or an acoustic waveguide. In one example, the gold electrode is deposited on the hypotenuse surface of the prism. In one aspect, the gold electrode is deposited in a region in which there is no interference with the illumination beam.

At operation 1970, the piezoelectric element is then affixed directly to the gold electrode on the prism using a thin layer of conductive silver paste. At operation 1980, wires are connected to the piezoelectric element to read the photoacoustic signals. At operation 1990, a protective layer (e.g., a thin parylene layer) is deposited onto the transducer element. As shown in the zoomed-in-view in FIG. 24, the structure of the piezo-element includes a silver matching layer and three gold electrodes. The first and second gold electrodes on both sides of the PMN-pt were used for poling the PMN-PT crystal. The third gold electrode on the surface of the matching layer is used to connect the gold electrode on the prism to form the circuit as the piezo-element may be connected by a double-shield coax cable. The inner conductor is connected with the E-solder conductive backing, and the copper braid is connected on the gold electrode on the prism (on the side of the piezo-element) to connect the ground side of the piezo-element.

(B) Calibration method and Calibrated Virtual Transducers

According to various embodiments, a PACTER system may only need a one-time universal calibration as discussed in Section V(B). With these embodiments, a PACTER system may be calibrated one time, e.g., at a factory before being passed onto the customer.

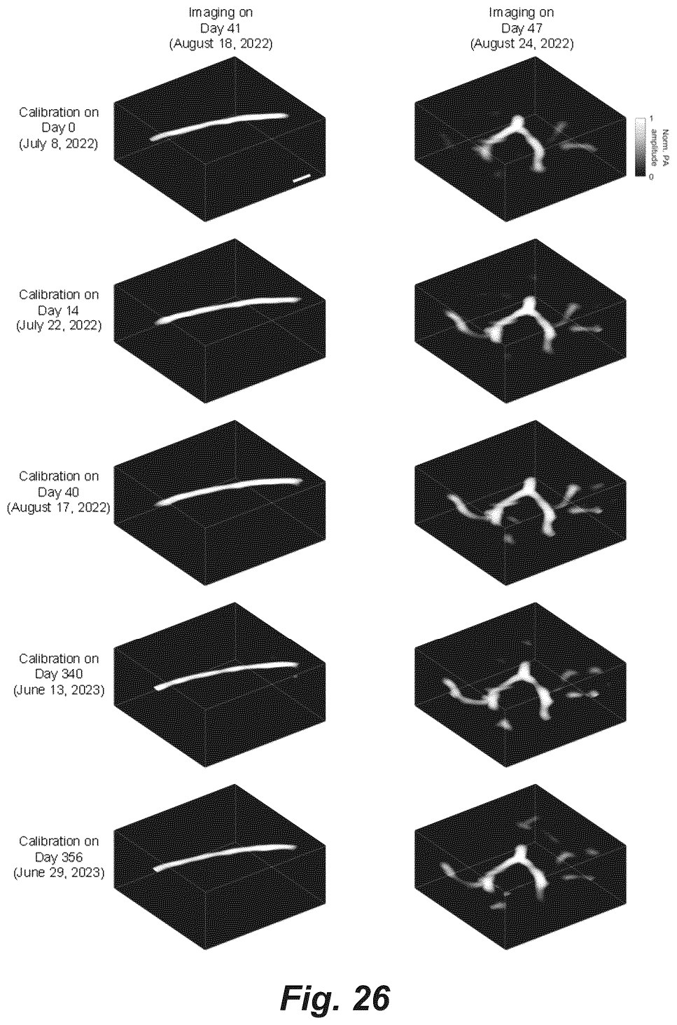

FIG. 26 depicts reconstructed PACTER images using calibration data acquired at different times over the course of a year with the PACTER system 300 in FIG. 3. Denoting Jul. 8, 2022 as day 0, the calibration data was acquired on days 0, 14, 40, 340, and 356. All of them can be used to reconstruct the imaging data acquired on days 41 and 47, which were acquired from a tube filled with bovine blood and the thenar vasculature of a human, respectively. The calibration data acquired over the course of a year could all be used for image reconstruction, which indicates that a single calibration dataset can be effective for at least a year.

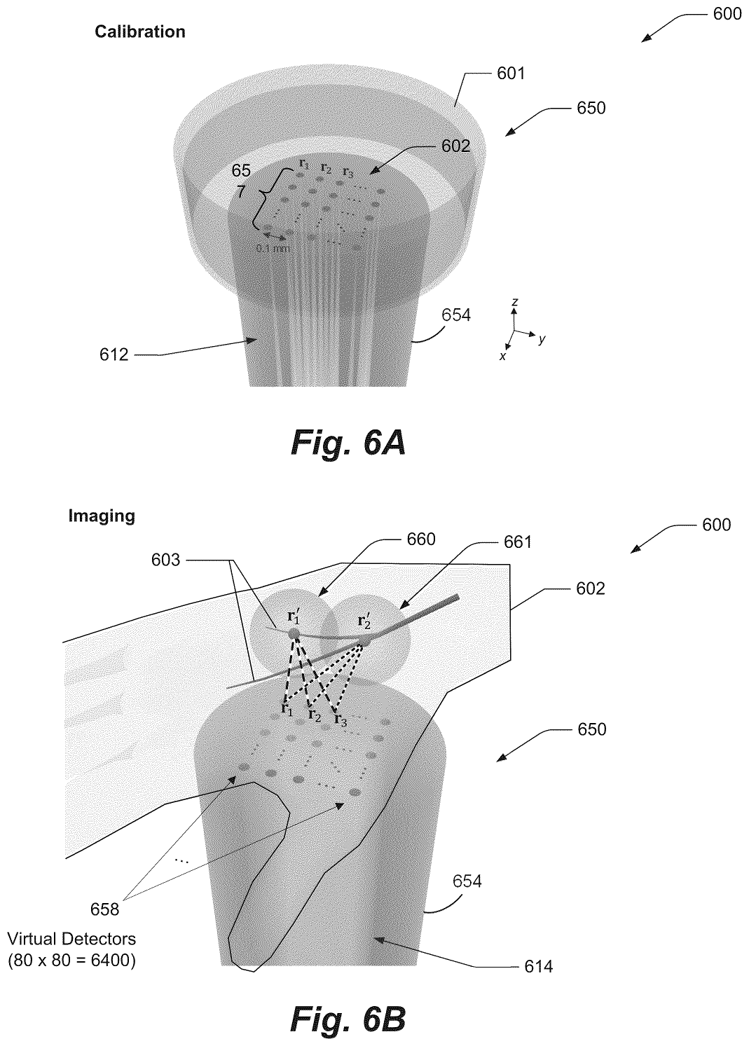

FIG. 6A depicts a schematic illustration of an ergodic relay 650 of a PACTER system 600 in a calibration configuration, according to an embodiment. PACTER system 601 may include one or more components of PACTER system 300 in FIG. 3. Ergodic relay 650 includes a prism (e.g., prism 352 in FIG. 3) and an optical rod 654 (e.g., silica rod). In this example, a proximal end of the optical rod 654 is fused to the prism. The illustrated example is shown at an instant during a calibration procedure during which a calibration target 601 is located in acoustic communication (e.g., with an acoustic medium) with a distal end of the optical rod 654. During the calibration procedure, one or more scanning devices such as one or more translation stages (e.g., first motorized stage 337 and second motorized stage 338 in FIG. 3) are implemented to steer a focused laser beam across to different locations (n×n array) over a field-of-view 602 of the calibration target 601. As shown, focused laser beams 612 are propagated through optical rod 654 to calibration target 601. The illustrated example depicts a rectangular n×n array (e.g., 80×80 array) of calibration pixels 657 as dots. The calibration step size used in the calibration procedure may be, e.g., 0.1 mm. The n×n array of calibration pixels 657 corresponds to N (N=n×n) calibrated virtual transducers. For example, a 80×80 array of calibration pixels corresponds to 6,400 calibrated virtual transducers. In the illustrated example, r₁, r₂, r₃ are the positions of three calibrated virtual transducers of the plurality of calibrated virtual transducers. Arrays of different dimensions and types (e.g., circular array) and/or calibration step sizes can be used according to other implementations.

FIG. 6B depicts a schematic illustration of ergodic relay 650 of PACTER system 600 in FIG. 6A in an imaging configuration in which palmar vessels 603 of a human hand 602 are being imaged, according to an embodiment. In the imaging configuration, PACTER system 600 may include one or more components (e.g., first microlens array 435 and second microlens array 436) of PACTER system 400 in FIG. 4. For example, the imaging procedure is performed using the calibrated virtual transducers 658 determined during the calibration procedure depicted in FIG. 6A. As shown in FIG. 6B, a homogenized beam 614 is used for widefield illumination. r′₁ and r′₂ are the positions of two source points in the palmar vessels. Sphere 660 and sphere 661 denote the photoacoustic (PA) waves generated by the two source points. The calibrated virtual transducers 658 capture the photoacoustic signals from r′₁ and r′₂ with different time delays, indicated by the long dashed lines from r′₁ and the short dashed lines from r′₂.

In the calibration procedure depicted in FIG. 6A, a focused laser beam is scanned across the field-of-view (FOV) 602 of calibration target 601 depicted as a plurality of focused laser beams 612 at a plurality of scanning positions at calibration pixels 657. The focused laser beam is scanned in n×n steps, e.g., in 80 by 80 steps with a step size of, e.g., 0.1 mm. During the calibration procedure, a data acquisition system of the PACTER system records a calibration signal at each of the scanning positions of the n×n array of calibration pixels 657 corresponds to the N (N=n×n) calibrated virtual transducers. The calibration signals obtained by scanning the light beam to n×n locations across the field-of-view 602 at the 2D plane in FIG. 6A can be used as n×n virtual transducers for 3D reconstruction because (1) the PACTER signals are object-independent, and (2) the calibration signals are generated at the bottom of the 3D imaging volume at the interface between the glass rod and the. For example, for an 80×80 array of calibration pixels, 80×80=6400 virtual transducers may be used for 3D reconstruction.

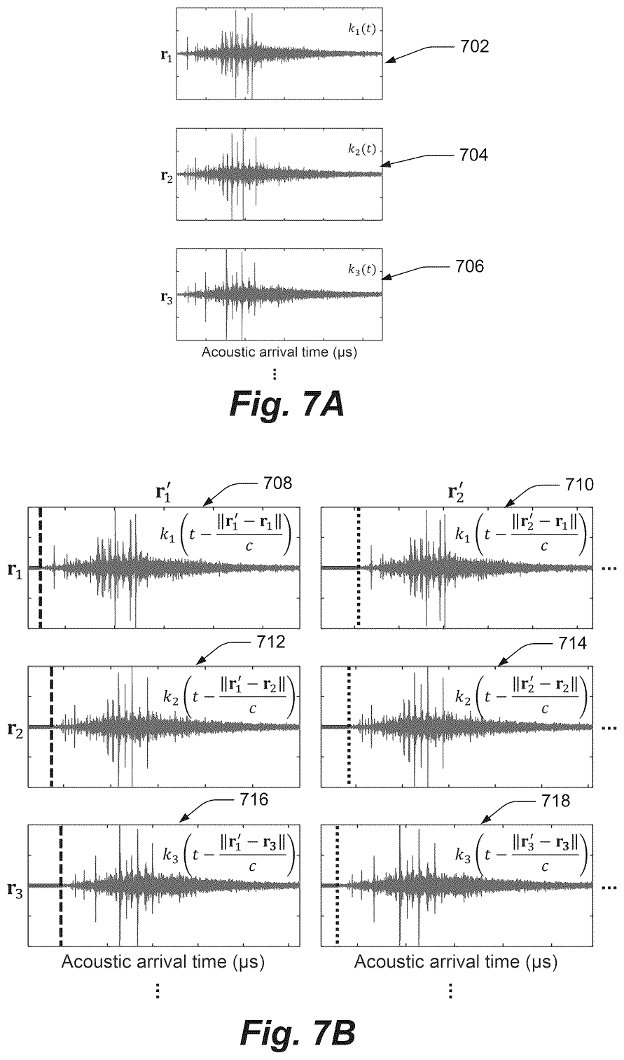

When source points 660, 661 in the 3D volume (r′ₘ, m=1, 2, . . . ) are illuminated by a light pulse, the photoacoustic signals they generate propagate to the calibrated virtual transducers (rₙ, n=1, 2, . . . ) after time t_{m,n}=∥r′ₘ−rₙ∥/c, where c is the speed of the sound in the medium. Then, these photoacoustic signals would follow the same acoustic path inside the ergodic relay 650 to the ultrasonic transducer as that of the calibration signals. From the transducer's perspective, compared with the calibration signal kₙ(t) acquired at In, the signal from the source point r′ₘ relayed through rₙ is proportional to kₙ(t) delayed by t_{m,n}, i.e., kₙ(t−∥r′ₘ−rₙ∥/c). FIG. 7A depicts plots 702, 704, and 706 of respective PACTER calibration signals, k₁(t), k₂(t), and k₃(t), of the calibrated virtual transducers corresponding to r₁, r₂, and r₃ in FIG. 6A. FIG. 7B depicts plots 708, 710, 712, 714, 716, and 718 of PACTER signals from the widefield imaging includes photoacoustic signals from source points r′₁ and r′₂, which are essentially calibration signals k₁ (t), k₂(t), and k₃(t) delayed according to the distance between the calibrated virtual transducer and the source point. The signal is modulated by both p_{0,m}, the initial pressure at r′n, and a weighting factor dependent on the angle and distance. Accordingly, the reconstruction method discussed in Section III(C)(1) can be used to reconstruct the initial pressure in the 3D volume. In certain embodiments, a forward model reformulated through temporal convolution and implemented using fast Fourier transformation discussed in Section V(C) can be used. The reformulated model implemented using fast Fourier transformation may increase computational efficiency by 9,100 times.

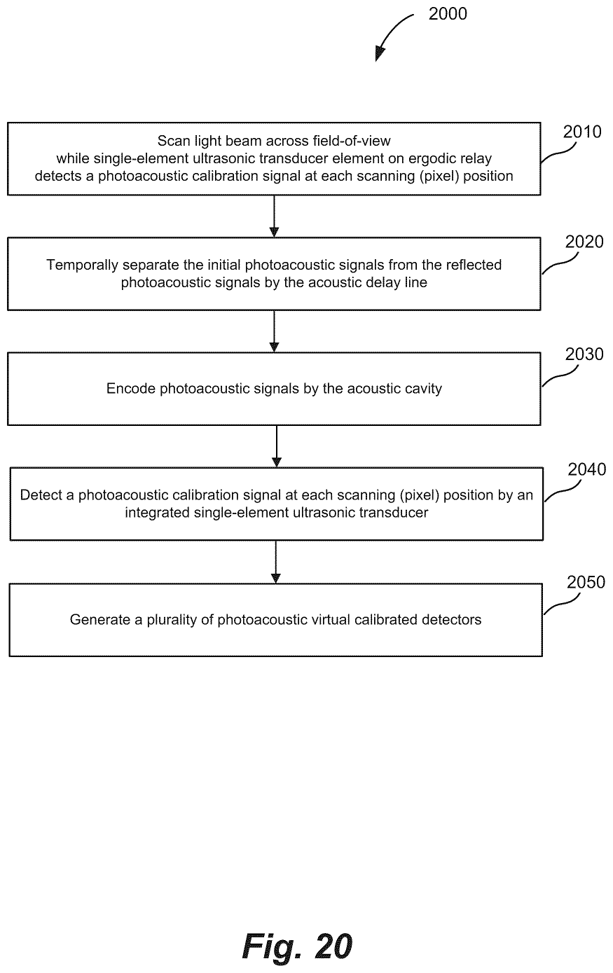

FIG. 20 depicts a flowchart 2000 of a PACTER method of calibrating a PACTER system, according to an embodiment. At operation 2010, a light source or light sources are triggered to provide pulses of light and a focused (illumination) light beam is propagated to the acoustic cavity of the ergodic relay. The focused illumination beam is scanned across a field-of-view of a calibration target (calibration field-of-view) placed in acoustic communication with the ergodic relay, for example, at a distal end of the acoustic delay line. A scanning device such as one or more translation stages (e.g., first motorized stage 337 and second motorized stage 338 in FIG. 3) steers the focused light beam to a plurality of calibration pixel locations (n×n array) over the calibration field-of-view. Any suitable step size may be used such as, e.g., 0.1 mm. Any suitable number of calibration pixel locations may be used such as, e.g., 50×50, 200×200, 500×500. Via the photoacoustic effect, photoacoustic waves are generated at the calibration pixel locations and photoacoustic waves are propagated to the acoustic delay line.

At operation 2020, at each light pulse the acoustic delay line (e.g., optical rod) temporally separates the initial photoacoustic signals from the reflected photoacoustic signals. For example, for an acoustic delay line with a length of 175 mm, the length of the delay will be 58 microseconds (175 mm×2 [round-trip]/speed of sound in glass [6000 m/s]).

At operation 2030, at each light pulse the acoustic cavity (e.g., prism) scrambles or encodes the photoacoustic signals received from the acoustic delay line. For each calibration pixel, the photoacoustic signal will be scrambled by the complex shape of the acoustic cavity and become a unique temporal pattern.

At operation 2040, while each calibration pixel location is illuminated by a focused light beam, a calibration photoacoustic signal is detected by the single-element ultrasonic transducer fabricated directly on a surface of the acoustic delay line of the ergodic relay. The single-element ultrasonic transducer may be fabricated in a region of the surface that will not be in interference with the focused illumination beam. In one aspect, the single-element ultrasonic transducer takes measurements at a rate of 100 MHz. In another aspect, the single-element ultrasonic transducer takes measurements at a rate of 250 MHz. In yet another aspect, the single-element ultrasonic transducer takes measurements at a rate of 500 MHz.

At operation 2050, the calibration photoacoustic signal detected at the plurality of calibration pixel locations are used as a corresponding plurality of photoacoustic virtual calibrated detectors. These virtual calibrated detectors are used in the image reconstruction method described in Section III(C)(1).

(C) PACTER Imaging method

### (1) PACTER Reconstruction Using Calibrated Virtual Transducers

In a PACTER system, the signal s(t) detected by the ultrasonic transducer at time t in a homogeneous medium is expressed as:

$$s ( t ) = {\sum }_{n = 1}^{N} {k}_{n} ( t ) * {\sum }_{m = 1}^{M} {p}_{0 , m} \frac{{1}_{[ 0 , {θ}_{1} ]} ( {θ}_{m , n} ) cos {θ}_{m , n}}{‖ {r}_{m}^{′} - {r}_{n} ‖} δ ( t - \frac{‖ {r}_{m}^{′} - {r}_{n} ‖}{c} ) , t \geq 0. \qquad ( Eqn . 1 )$$

Here, M and N are the numbers of the source points and the calibrated virtual transducers, respectively; kₙ(t) is the normalized impulse response from the calibration at the n- the virtual transducer; r′ₘ and rₙ are the locations of the m-th source point and the n-th virtual transducer, respectively; p_{0,m} is a value proportional to the initial pressure at r′ₘ; θ_{m,n} denotes the incidence angle satisfying

$$cos {θ}_{m , n} = \frac{( {r}_{m}^{′} - {r}_{n} ) \cdot n}{‖ {r}_{m}^{′} - {r}_{n} ‖}$$

with n being the normal vector of the calibration plane; θ₁ is the critical angle the ultrasonic refraction from water to fused silica; 1_{[0, θ}_{_{1]}} represents the indicator function defined in Eqn. 10; c is the speed of sound in the homogeneous medium; δ(t) denotes the delta function. To simplify the reconstruction task, it is assumed the object has a homogeneous speed of sound. However, to yield high-resolution, artifact-free PAT images, the inhomogeneities of the speed of sound in biological tissues should be taken into account (. A forward model and image reconstruction discussion that derives Eqn. 1 is provided in Section V(C).

Discretizing Eqn. 1 obtains the forward model:

$$s = H {p}_{0} , \qquad ( Eqn . 2 )$$

where s represents a vector of length L, P₀ denotes a vector of length M(=M₁M₂M₃) which consists of all voxels in a 3D image of size M₁×M₂× M₃, and H is the system matrix of size L×M. This forward model has a computational complexity of max{O(MN), O(NLlog₂L)}. To obtain an image from the signals s, the forward model is inverted by solving the regularized optimization problem:

$${\hat{p}}_{0} = {arg min}_{{p}_{0} ∈ {ℝ}^{M} , {p}_{0} \geq 0} {‖ {Hp}_{0} - s ‖}^{2} + λ {\vert {p}_{0} \vert}_{TV} . \qquad ( Eqn . 3 )$$

Here, |P₀|_{TV} denotes the total variation (TV) of the 3D image corresponding to p₀, and 1 is the regularization parameter. TV regularization aids in transforming an image into a new one with piecewise smoother structures, essentially constituting a form of sparseness. Utilizing TV regularization allows the incorporation of the piecewise smoothness of blood vessels into the iterative reconstruction, considerably stabilizing the iterations. Numerically, this optimization problem is solved through a Fast Iterative Shrinkage-Thresholding Algorithm (FISTA). An example of a FISTA algorithm can be found in Beck, A. & Teboulle, M. A, “fast iterative shrinkage-thresholding algorithm for linear inverse problems. SIAM,” Imaging Sci. 2, 183-202 (2009). An iteration number of 8 was chosen for the FISTA algorithm, considering the trade-off between the reconstruction image quality and the image reconstruction time. This choice was determined through a series of experiments, in which the impact of different iteration numbers was analyzed for both the quality of the reconstructed image and the computational time required for reconstruction. For a 3D volume comprising 80×80×120 voxels, the image reconstruction time was approximately 600 s running on a CentOS Linux 7 system with Intel® Xeon® Gold 6130 CPU @ 2.10 GHz.

### (2) Processing of Reconstructed PACTER Images

In some embodiments, the reconstructed images are processed. In one embodiment, the reconstructed images are first denoised using a 3D median filter (e.g., in the 3-by-3-by-3 neighborhood) and smoothed using a 3D Gaussian filter (e.g., with a 0.1-by-0.1-by-2 standard deviation kernel). A Hessian-matrix-based vesselness filter may be applied to the denoised images to improve the contrast of vascular structures in 3D. The vesselness-enhanced images (self-normalized) with a weighting factor of 0.8 is added back to the filtered images with a weighting factor of 0.2 and obtained the final images. The images may be rendered in 3D or in 4D (e.g., time lapse 3D) using various algorithms such as, e.g., the Imaris (Bitplane) software.

### Speed of Fluid Flow

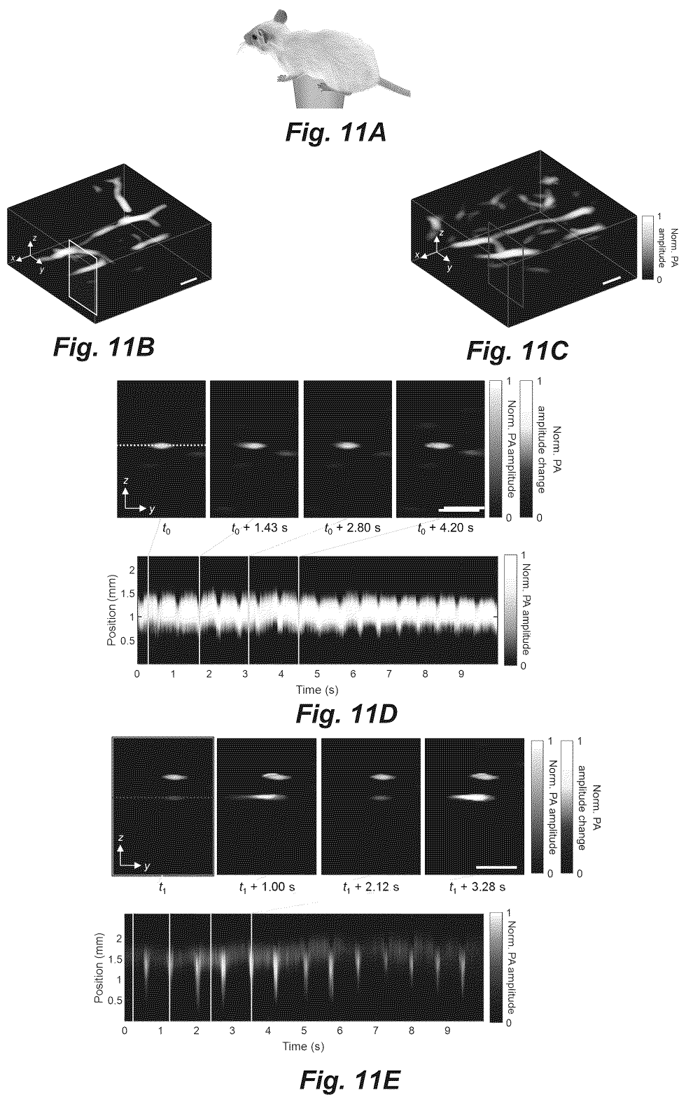

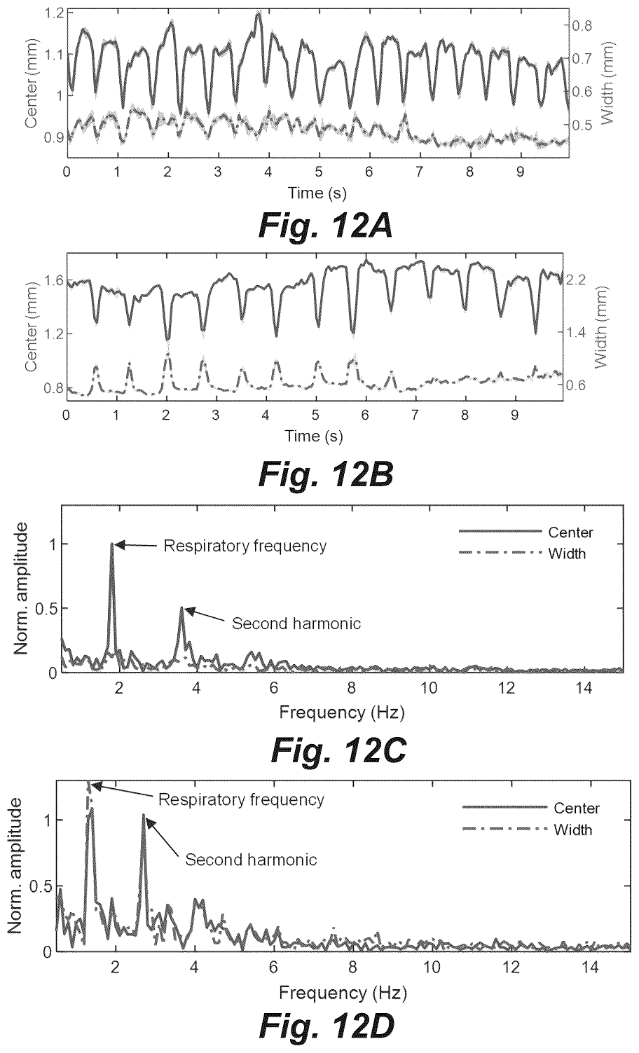

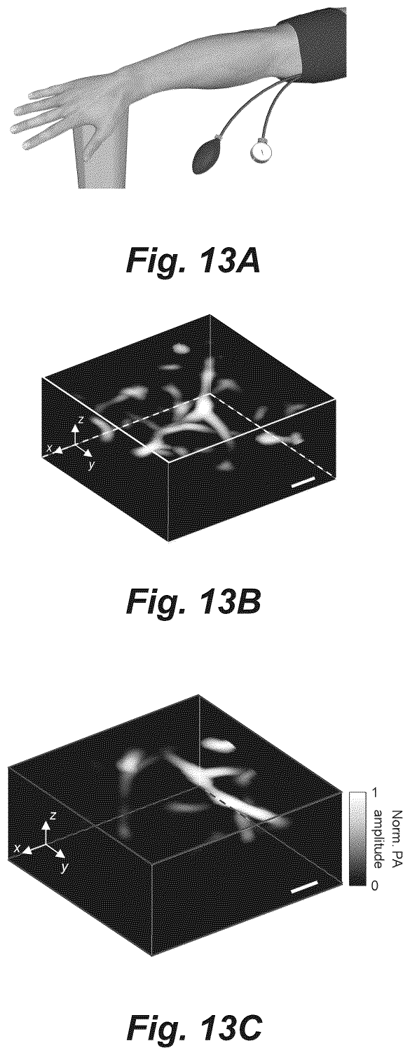

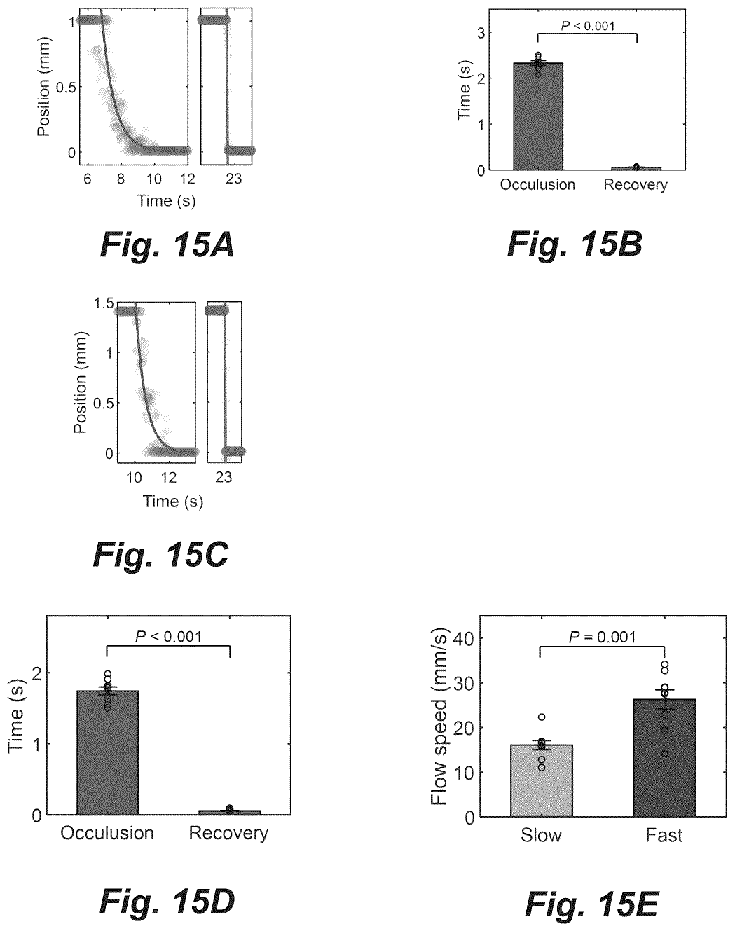

In one embodiment, the speed of fluid flow may be calculated from PACTER data by differentiating the PA amplitudes along a fluid path and fitting the relationship between the travelling distance of the fluid front over time. For example, the speed of bovine blood flushing through a tube may be calculated by differentiating PA amplitudes along the tube and fitting the relationship between the traveling distance of the blood front versus time. In FIGS. 11D-E and FIGS. 12A-D, for example, the vessels' profiles were fitted as a Gaussian function, exp(−(x−x₀)²/w²), where the center positions and widths of the vessels were estimated from x₀ and 2\sqrt{In2}w, respectively. The positions of the blood front along the blood vessels in FIGS. 13B-C, 14A-B, and 15A-E were obtained through thresholding the PA amplitude profiles. Denoting the total length of the blood vessel profile as Lₚ, the positions of the blood front along the blood vessels during the occlusion and recovery phases were fitted with dₒ(t)=a exp(−vₒt/Lₚ) and dᵣ(t)=−vᵣt+b, respectively, where a and b were constants, vₒ was the occlusion rate, and vᵣ was the blood flow speed. The durations of the occlusion and recovery phases were estimated by t₀=3Lₚ/vₒ and tᵣ=0.95Lₚ/vᵣ, respectively, which were the time it took for the blood front to propagate 95%, i.e., 1− exp(−3), of Lₚ. To determine if the differences between tₒ and tᵣ were significant a Welch's (unequal variances)t-test was applied to determine the P values under the null hypothesis that the mean values of tₒ are not different from those of tᵣ. The same t-test was also performed to determine if the difference between the blood flow speeds in FIG. 15E was significant.

### (3) PACTER Imaging Method

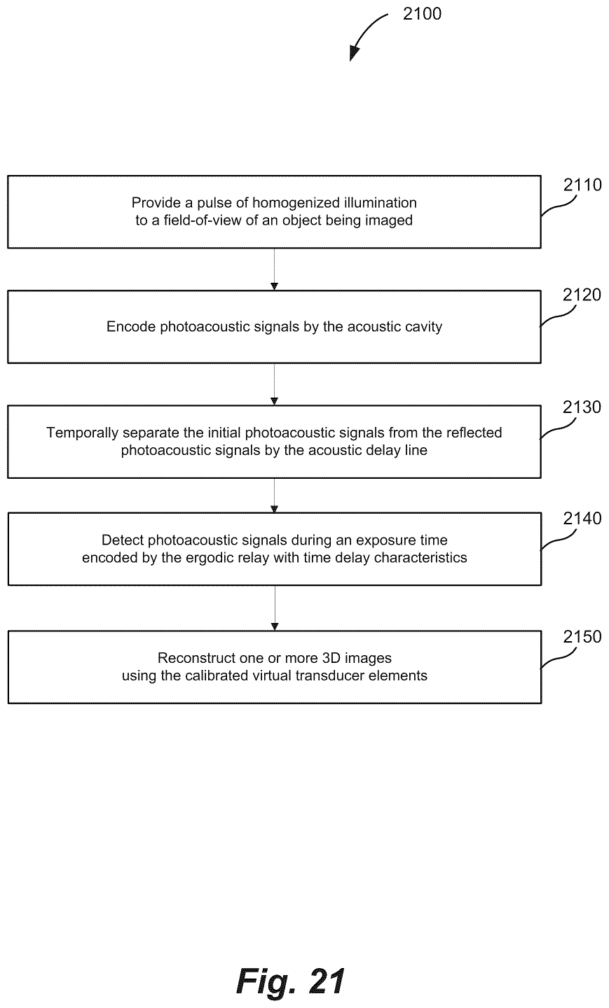

FIG. 21 depicts a flowchart 2100 of a PACTER imaging using a PACTER system such as PACTER system 100 in FIG. 1, according to an embodiment. At operation 1910, a light source or light sources is triggered to provide a pulse of light. A homogenizer converts incident light from the pulse of light to into a homogenized illumination pattern that is propagated to the acoustic cavity of the ergodic relay. The homogenized illumination pattern is propagated to a field-of-view of an object placed in acoustic communication with the ergodic relay, for example, at a distal end of the acoustic delay line. Via the photoacoustic effect, photoacoustic waves are generated at the object and photoacoustic waves are propagated to the acoustic delay line.

At operation 2120, at the acoustic delay line (e.g., optical rod) temporally separates the initial photoacoustic signals from the reflected photoacoustic signals. For example, for an acoustic delay line with a length of 175 mm, the length of the delay will be 58 microseconds (175 mm×2 [round-trip]/speed of sound in glass [6000 m/s]).

At operation 2130, at each light pulse the acoustic cavity (e.g., prism) scrambles or encodes the photoacoustic signals received from the acoustic delay line. The photoacoustic signal is scrambled by the complex shape of the acoustic cavity and becomes a temporal pattern that is a function (following Eqn. 1) of the calibration pixel signals captured at operation 2030.

At operation 2140, while each the field-of-view of the object being imaged is illuminated by the homogenized illumination pattern, a photoacoustic signal is detected by a single-element ultrasonic transducer fabricated directly on a surface of the acoustic delay line of the ergodic relay. A DAQ records and digitizes the photoacoustic signal over an exposure time. The single-element ultrasonic transducer may be fabricated in a region of the surface that will not be in interference with the illumination beam. In one aspect, the single-element ultrasonic transducer takes measurements at a rate of 100 MHz. In another aspect, the single-element ultrasonic transducer takes measurements at a rate of 250 MHz. In yet another aspect, the single-element ultrasonic transducer takes measurements at a rate of 500 MHz.

At operation 2150, a 3D photoacoustic image is reconstructed using the recorded data from the encoded photoacoustic signal and the photoacoustic virtual calibrated detectors from a calibration procedure performed on the PACTER system. The recorded data and the virtual calibrated detectors are used in the image reconstruction method described in Section III(C)(1).

### IV. Demonstrations

(A) Spatiotemporal characterization of PACTER images

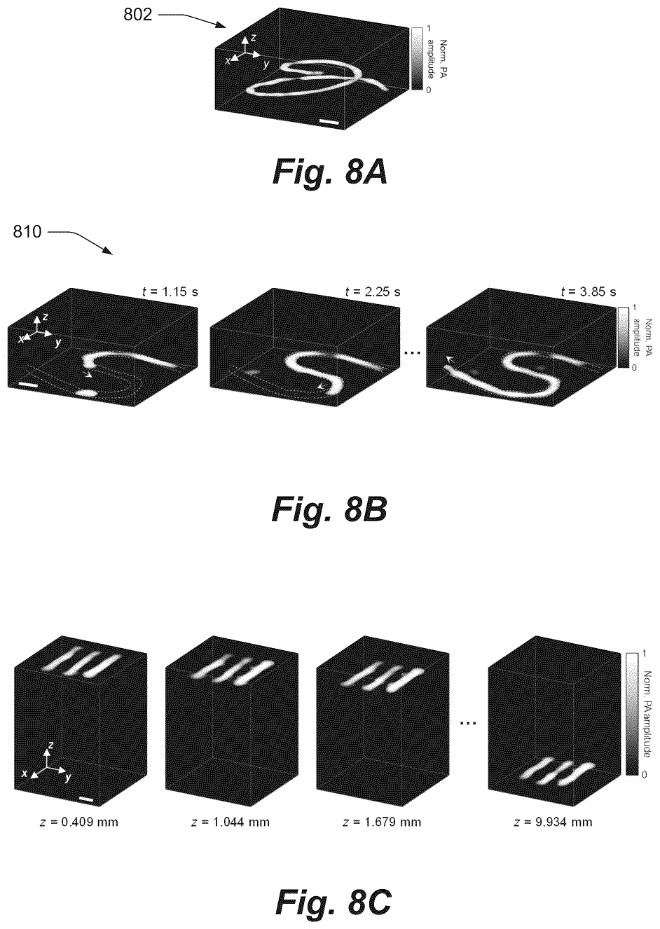

A PACTER system can be used to image the 3D structure of an object and the 4D dynamics of a moving object (e.g, fluid flow) when illuminated by multiple light pulses. For example, using the signals acquired by a single-element ultrasonic detector, the PACTER system 400 of FIG. 4 was used to image the 3D structure of a curved black wire with a single laser pulse as shown by image 802 in FIG. 8A and to image the 4D dynamics of bovine blood flushing through an S-shaped tube when the tube was illuminated by multiple laser pulses as illustrated by the images 810 in FIG. 8B. FIG. 8A is a 3D PACTER image of a curved black wire acquired by the PACTER system 400 in FIG. 4. FIG. 8B are snapshots taken by PACTER system 400 in FIG. 4 at time instances t₀=1.15s, t₁=2.25s, . . . t_{N}=3.85s of a 4D PACTER implementation showing bovine blood flushing through an S-shaped tube.

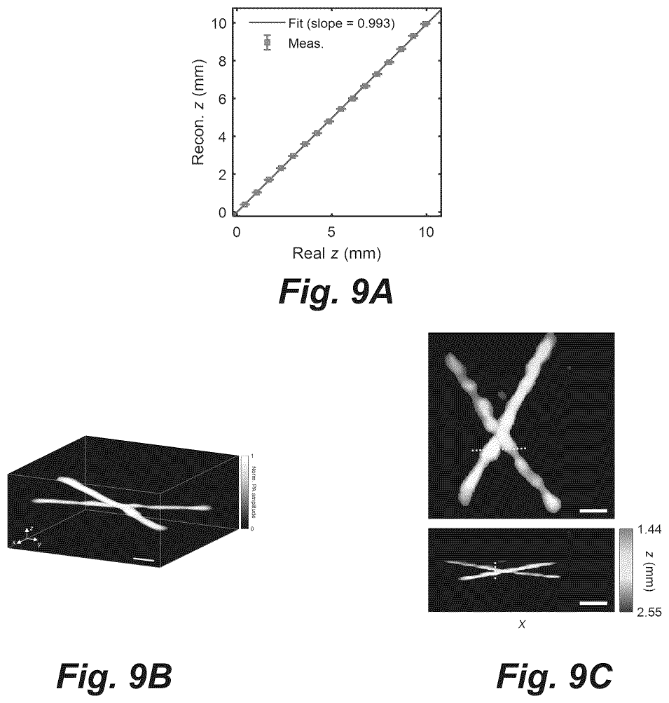

Second, the PACTER system 400 in FIG. 4 was used to image a thin object (three bars printed with black ink on a transparent film) in water, whose z positions were precisely controlled and measured by a linear translation stage. The object was imaged at multiple z positions, the 3D volumes reconstructed generating images, and the z positions in the reconstructed volumes were compared with the real ones. FIG. 8C depict 3D images taken by PACTER system 400 in FIG. 4 of the three bars printed with black ink on a transparent film. In each image, the object was placed at different z positions: z₀=0.409 mm, z_{1=1.044} mm, . . . z_{N}=9.934 mm. FIG. 9A is a plot of the PACTER reconstructed z-positions versus real (measured) z positions of the three bars in FIG. 8C. The measurement results are plotted as means±standard errors of the means (n=1,980). The solid line represents a linear fit. As shown in FIG. 9A, the reconstructed and real z positions were linearly related (R²=1.000) with a slope (0.993) close to 1, demonstrating that a PACTER system can accurately reconstruct the 3D objects in the axial (z) direction.

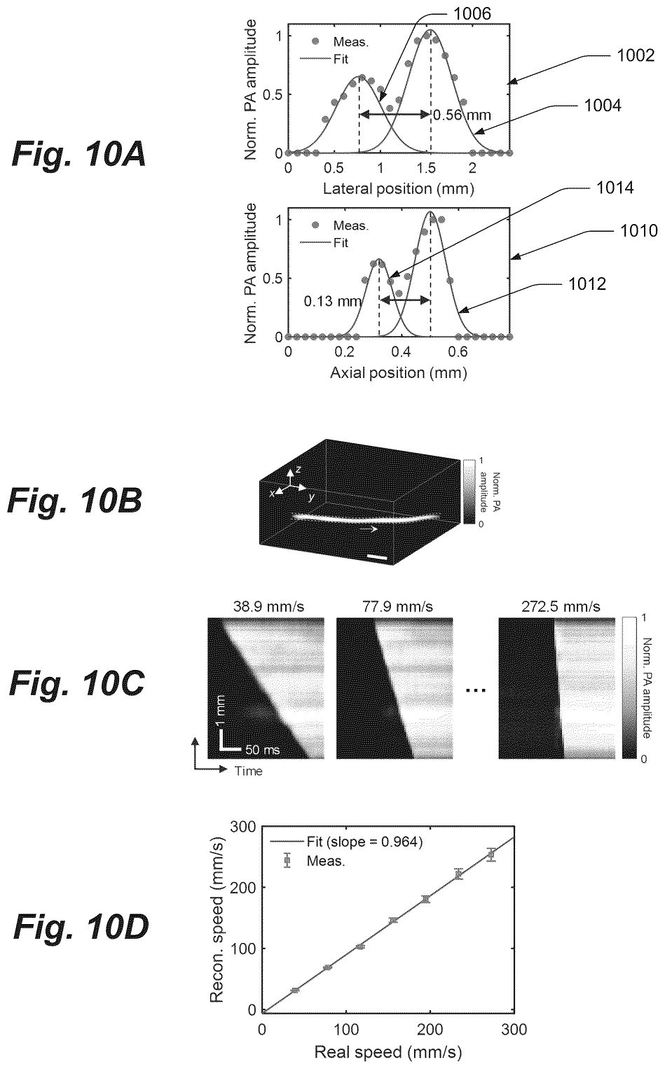

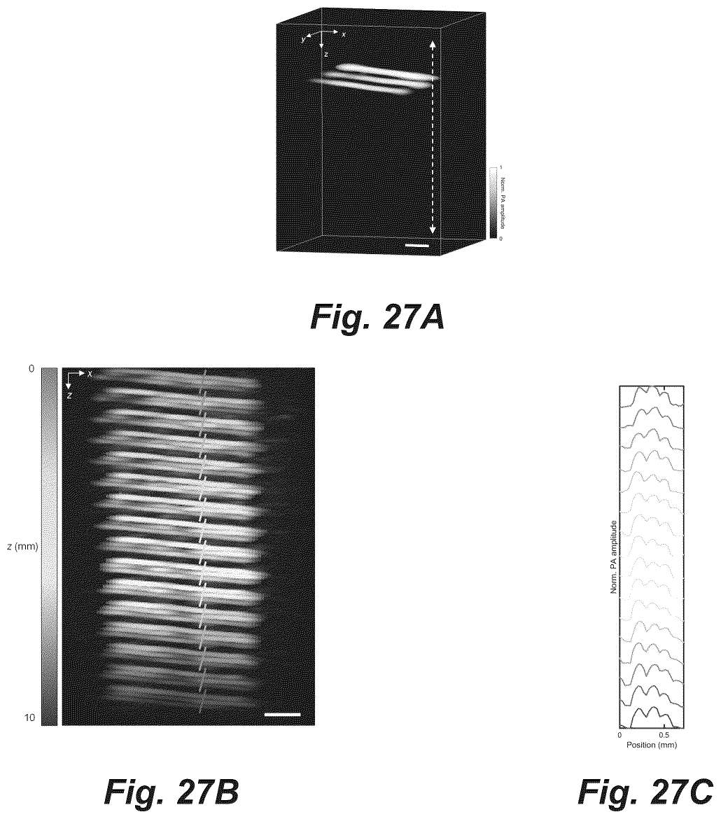

To quantify the spatial resolution of the PACTER system 400 in FIG. 4, two human hairs embedded in an agarose block were imaged. FIG. 9B is a 3D PACTER image of the two crossing human hairs in agarose taken by PACTER system 400. The two hairs were intentionally positioned such that they were in close contact with each other, forming a crossing pattern that could be seen in both z- and y-projections. FIG. 9C includes two images showing the crossing pattern. The top image is a maximum z-projection of the 3D volume of the 3D PACTER image in FIG. 9B. The bottom image is a maximum y-projection of the 3D PACTER image in FIG. 9B. The z positions of the object are color-encoded. Defining the spatial resolution as the minimum distance that can distinguish the peaks of the two hairs, the lateral and axial resolutions of the PACTER system 400 were found to be 0.56 mm and 0.13 mm, respectively. FIG. 10A depicts profiles 1002, 1010 along the dashed lines in FIG. 9C denoted by gray dots. The curves 1004, 1006, 1012, 1014 represent two-term Gaussian fits. The double-sided arrows denote the minimum distances that separate the two human hairs and the associated lateral and axial resolutions. The anisotropic spatial resolutions along the lateral and axial directions are related to the image formation process in a PACTER system as discussed in Section V(D) and (E). By imaging a thin object placed at different z positions, the resolution of the PACTER system 400 was found to be not sensitive to the imaging depth as shown in FIGS. 27A-C. FIG. 27A depicts a 3D PACTER image of three bars printed with black ink on a transparent film. The dashed line indicates that the object is translated along the z-axis to be imaged at different depths. FIG. 27B depicts a summation of the maximum y-projections of the 3D volumes like FIG. 27A where the object is placed at different depths. The z-positions of the object are color-encoded. FIG. 27C depicts a plot of profiles along the solid lines in FIG. 27B. The colors represent the depths of the object. Scale bars are 1 mm.

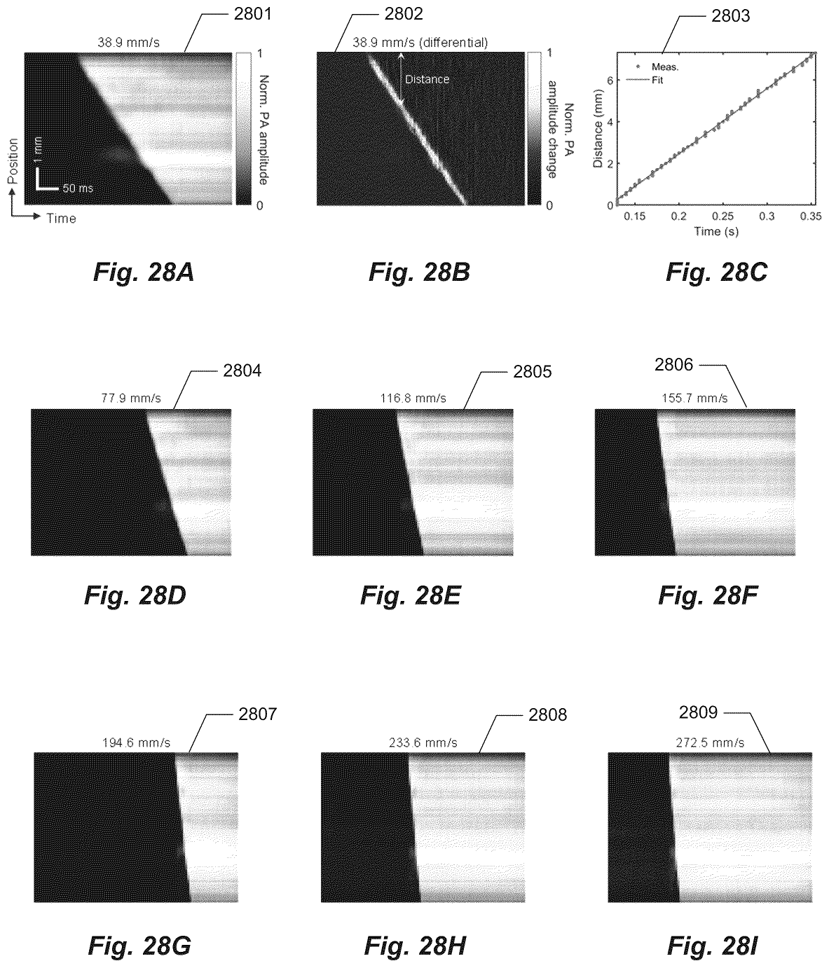

To demonstrate that a PACTER system can be used to image 4D dynamics, i.e., time-lapse movements of 3D objects, PACTER system 400 in FIG. 4 was used to capture 4D images of bovine blood flushing through a tube at different speeds precisely controlled by a syringe pump. FIG. 10B depicts a 3D PACTER image of bovine blood flushing through a tube as imaged by PACTER system 400 in FIG. 4. The white arrow indicates the flushing direction. Based on the reconstructed 4D images, the PA amplitudes along the tube (1D images) were plotted over time. FIG. 10C depicts photoacoustic amplitudes along the tube in FIG. 10B versus time, when the blood flushes through the tube at different speeds. The speeds of the blood flow were calculated and compared with the real speeds set by the syringe pump. FIG. 28A depicts a first image 2801 of PA amplitudes along the tube (1D image) flushed by the blood with a speed of 38.9 mm/s versus time. FIG. 28B depicts a second differential image 2801 (difference between adjacent voxels along the tube in first image 2801 versus time) showing the distance the blood front travels over time. FIG. 28C depicts a plot of traveling distance of the blood front plotted over time. FIG. 28D depicts an image of PA amplitudes along the tube (1D images) flushed by the blood with different speeds versus time. FIG. 28E depicts an image of PA amplitudes along the tube (1D images) flushed by the blood with different speeds versus time. FIG. 28F depicts an image of PA amplitudes along the tube (1D images) flushed by the blood with different speeds versus time. FIG. 28G depicts an image of PA amplitudes along the tube (1D images) flushed by the blood with different speeds versus time. FIG. 28H depicts an image of PA amplitudes along the tube (1D images) flushed by the blood with different speeds versus time. FIG. 28I depicts an image of PA amplitudes along the tube (1D images) flushed by the blood with different speeds versus time. FIG. 10D depicts a plot of the speeds of the blood flow quantified from the reconstructed PACTER images in FIGS. 28A-I versus the real speeds of the blood flow set by the syringe pump along the tube shown in FIG. 10B. A linear relationship (R²=0.999) between the reconstructed and real speeds with a slope (0.964) close to 1 can be observed, demonstrating that a PACTER system is capable of 4D imaging, faithfully reconstructing the dynamics of 3D objects over time. Empowered by the imaging speed of up to a thousand volumes per second, PACTER could resolve the high-speed dynamics of the blood flushing through the tube at 272.5 mm/s in 3D, with, e.g., a temporal resolution of 1 ms for a laser repetition rate of 1 kHz or a temporal resolution of 1 us for a laser repetition rate of 1 MHz.

(B) 4D In Vivo Imaging of Mouse Hemodynamics with PACTER

Enabled by the capability of noninvasive, label-free, and ultrafast 3D imaging, PACTER techniques may be suitable for monitoring hemodynamics in vivo. The PACTER system 400 in FIG. 4 was used to demonstrate that PACTER techniques can be used to monitor vital signs such as breathing in, for example, small animals.

The PACTER system 400 in FIG. 4 was used to image the hemodynamics of the abdominal regions of mice. The PACTER system 400 in FIG. 4 was pre-calibrated using bovine blood. FIG. 11A is a schematic illustration of a mouse PACTER imaging configuration where the mouse abdomen is located directly on a distal end of an optical rod of an ergodic relay of a PACTER system. With a single laser pulse, the PACTER system 400 was used to reconstruct the abdominal vasculature in 3D of two mice. FIG. 11B depicts a 3D PACTER image of the abdominal vasculature of a first mouse (“Mouse 1”) captured by PACTER system 400 in FIG. 4. FIG. 11C depicts a 3D PACTER image of the abdominal vasculature of a second mouse (“Mouse 2”) captured by PACTER system 400 in FIG. 4.

When multiple laser pulses were used, PACTER system 400 was able to capture the 4D dynamics of the blood vessels. Based on the 4D PACTER datasets, individual blood vessels were isolated from the cross sections of the 3D volumes were isolated and visualized their motions and structural changes were visualized. FIG. 11D depicts cross-sectional 2D images (top) corresponding to the rectangle in FIG. 11B of mouse 1 at four different time instances from the 4D PACTER datasets. t₀=0.28 s, t₁=0.26 s and a plot (bottom) of the photoacoustic amplitudes along the dashed line in the first cross-sectional 2D image versus time, wherein the time instances are labeled with vertical lines. FIG. 11E depicts cross-sectional 2D images (top) corresponding to the rectangle in FIG. 11C of mouse 2 at four different time instances from the 4D PACTER datasets. t₀=0.28 s, t₁=0.26 s and a plot (bottom) of the photoacoustic amplitudes along the dashed line in the first cross-sectional 2D image versus time, wherein the time instances are labeled with vertical lines. FIG. 12A depicts a plot of the center positions (solid curves) and widths (dash-dotted curves) of the vessels versus time, based on fits from the cross-sectional 2D images of FIG. 11D. FIG. 12B depicts a plot of the center positions (solid curves) and widths (dash-dotted curves) of the vessels versus time, based on fits from the cross-sectional 2D images of FIG. 11D.

By recording the time-lapse changes of the center positions and widths of the blood vessels, the respiratory motion could be tracked and identified. Using Fourier analysis, it was found that the center position of the blood vessel of mouse 1 fluctuated periodically, exhibiting a respiratory frequency of 1.8 Hz, whereas the width of the vessel was relatively stable. In comparison, a respiratory frequency of 1.4 Hz could be observed from both the center position and width of the blood vessel of mouse 2. FIG. 12C depicts a plot of Fourier transforms of the center positions and widths of the vessels in FIG. 12A showing the respiratory frequency from the vessel center positions only. FIG. 12D depicts a plot of Fourier transforms of the center positions and widths of the vessels in FIG. 12B showing the respiratory frequency from both the vessel center positions and widths.

(C) 4D In Vivo Imaging of Hemodynamics in Human Hands with PACTER

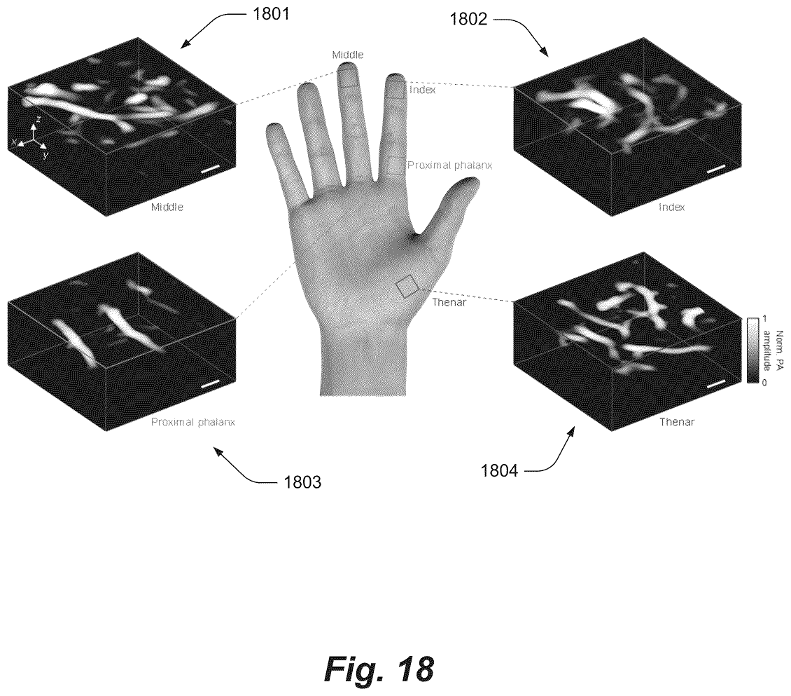

To demonstrate PACTER techniques being used to monitor hemodynamics in humans, PACTER system 400 in FIG. 4 was employed to image the hand vasculature of two human subjects (“Subject 1”) and (“Subject 2”). The PACTER system 400 in FIG. 4 was pre-calibrated using bovine blood. Different regions of the hand, e.g., fingers, proximal phalanx, and thenar regions, were imaged independently as the subjects moved their hands to align those regions with the ergodic relay. FIG. 18 depicts a PACTER image 1801 of a middle finger, a PACTER image 1802 of an index finger, a PACTER image 1803 of a proximal phalanx region, and a PACTER image 1804 of a thenar region.

The thenar vasculature was imaged and responses to cuffing, which was induced by a sphygmomanometer wrapped around the upper arm was imaged. FIG. 13A is a schematic illustration of a human hand imaging configuration where the hand is located directly on a distal end of an optical rod of an ergodic relay of a PACTER system 400 and a sphygmomanometer is wrapped around the upper arm. For imaging the vasculature in human hands, after applying ultrasound gel, the hands were placed on top of the ergodic relay 150. For imaging the human hand hemodynamics in response to cuffing, a sphygmomanometer was wrapped around the participants' upper arm as shown in FIG. 13A. To induce blood vessel occlusion, the sphygmomanometer was inflated to high pressure (200 mmHg), maintained for a short time (˜15 s), and then quickly released; the total imaging time was 30 s.

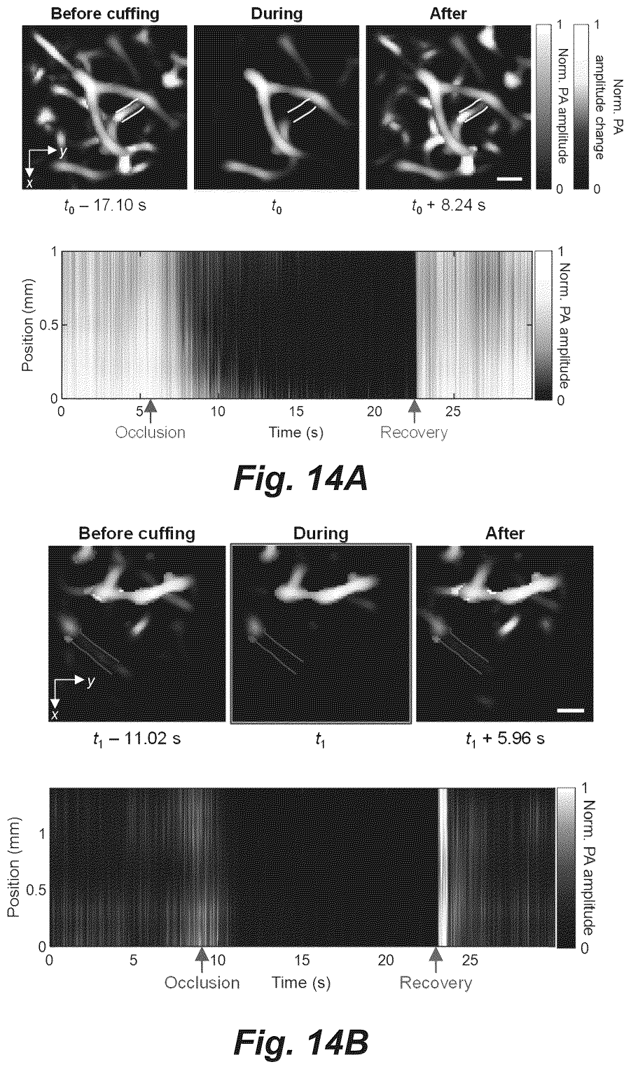

Using the PACTER system 400, the thenar vasculature of Subject 1 and Subject 2 was imaged in 3D with single laser pulses and the 4D dynamics of the blood vessels in response to cuffing was reconstructed. FIG. 13B depicts a 3D PACTER image of the thenar vasculature of Subject 1. FIG. 13C depicts a 3D PACTER image of the thenar vasculature of Subject 2. FIG. 14A depicts the maximum amplitude projections of the 3D volumes from the 4D PACTER datasets along the z axis in FIG. 13B for Subject 1 at the time instances before, during, and after cuffing. t₀=17.44 s, t₁=19.02 s for Subject 1 and PA amplitudes along the vessels (1D images) in FIG. 13B versus time, where the time instances are labeled with vertical lines. FIG. 14B depicts the maximum amplitude projections of the 3D volumes from the 4D PACTER datasets along the z axis in FIG. 13C for Subject 2 at the time instances before, during, and after cuffing. t₀=17.44 s, t₁=19.02 s for Subject 1 and PA amplitudes along the vessels (1D images) in FIG. 13C versus time, where the time instances are labeled with vertical lines. The arrows indicate peak responses in the occlusion and recovery phases, respectively. FIG. 15A depicts a plot of the positions (solid circles) of the blood front along the blood vessel during the occlusion (left) and recovery (right) phases in FIG. 14A. The curve in the left plot is an exponential fit with an occlusion rate of 1.3±0.1 m/s, and the curve in the right plot is a linear fit showing the blood flow speed of 16.1±3.1 m/s. FIG. 15B depicts a plot of the comparison between the durations of the occlusion and recovery phases in FIG. 14B. FIG. 15B depicts a plot of the comparison between the durations of the occlusion and recovery phases in FIG. 14A. FIG. 15C depicts a plot of positions (solid circles) of the blood front along the blood vessel during the occlusion (left) and recovery (right) phases in FIG. 14B. The left curve is an exponential fit with an occlusion rate of 2.4±0.2 m/s, and the right curve is a linear fit showing the blood flow speed of 26.3±6.4 m/s. FIG. 15D depicts a bar graph of a comparison between the durations of the occlusion and recovery phases in FIG. 14B. FIG. 15E depicts a bar graph of a comparison between the blood flow speeds during recovery in FIGS. 14A and 14B.

As shown in the maximum amplitude projections of the 4D datasets shown in in the top portions of FIGS. 14A and 14B, whereas some blood vessels exhibited a non-decreased PA amplitude throughout the experiment, the other vessels showed a decreased PA amplitude after cuffing due to the occlusion of blood flows; when the cuffing was released, the blood flows recovered, and the PA amplitude was rapidly restored as shown in the bottom portions of FIGS. 14A and 14B. The different hemodynamics of these two types of blood vessels in response to cuffing indicate their distinct roles in the circulatory system: the blood vessels with non-decreased and decreased PA amplitudes could be venous and arterial, respectively, agreeing with the observations reported in other cuffing-based studies. With the capability to simultaneously image both arterial and venous blood in vivo, PACTER provides additional benefits over conventional pulse oximetry, which can only monitor arterial blood without spatial resolution. Because PA amplitudes have 100% sensitivity to optical absorption, the 4D hemodynamics imaged by PACTER revealed the real-time changes in the blood vessels in response to cuffing, and the linear position of the blood front during the recovery phase could be used to measure the blood flow speed.

For Subject 1, the occlusion rate of the vessel was found to be 1.3+0.1 m/s, significantly slower than the blood flow speed of 16.1+3.1 m/s extracted from the recovery phase as shown in FIGS. 15A and 15B. For Subject 2, the occlusion rate and the blood flow speed of the vessel were found to be 2.4+0.2 m/s and 26.3+6.4 m/s, respectively as shown in FIGS. 15C and 15D. exhibiting a greater blood flow speed compared with participant 1 as shown in FIG. 15E. Taken together, it was demonstrated that PACTER techniques may be used to monitor the hemodynamics in human, including the consistent responses of thenar vasculature to cuffing, and capture the variability in blood flow speeds.

(D) 4D In Vivo Imaging of Hemodynamic Changes in Human Foot Vessels with PACTER

Imaging of hemodynamics in the lower extremities, specifically in the human feet, plays a pivotal role in the diagnosis, treatment, and prevention of peripheral vascular diseases and diabetes.

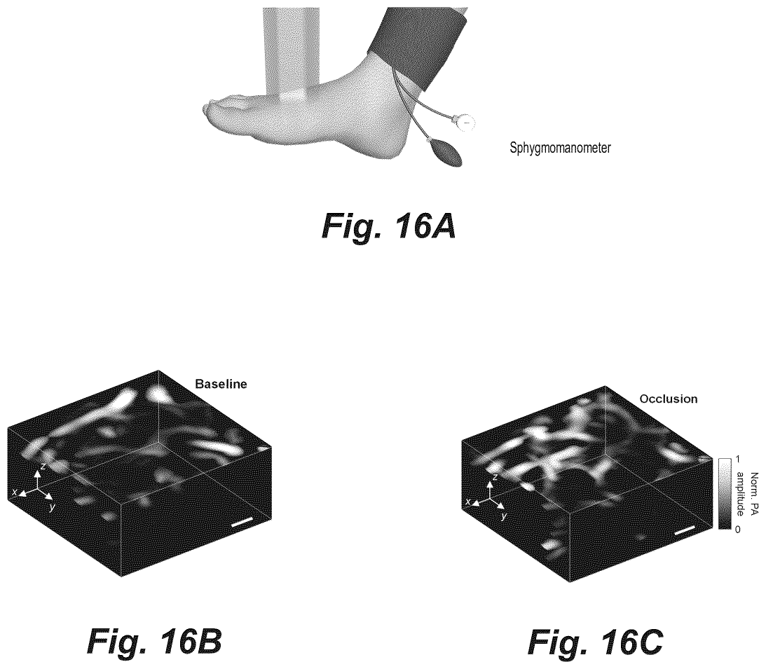

To evaluate the clinical applicability of PACTER, PACTER system 400 in FIG. 4 was used to image the hemodynamic changes in human foot vessels in response to vascular occlusion, which was induced by a sphygmomanometer wrapped around the participant's leg. The PACTER system 400 in FIG. 4 was pre-calibrated using bovine blood. FIG. 16A is a schematic illustration of a human foot PACTER imaging configuration where the foot is located in direct contact with a distal end of an optical rod of an ergodic relay of a PACTER system 400. For imaging hemodynamic changes in human foot vessels, a sphygmomanometer was wrapped around the participant's leg as shown in FIG. 16A. To induce vascular occlusion, the sphygmomanometer was inflated to high pressure (200 mmHg), maintained for 15 s to 20 s, and then quickly released; the total imaging time was 30 s.

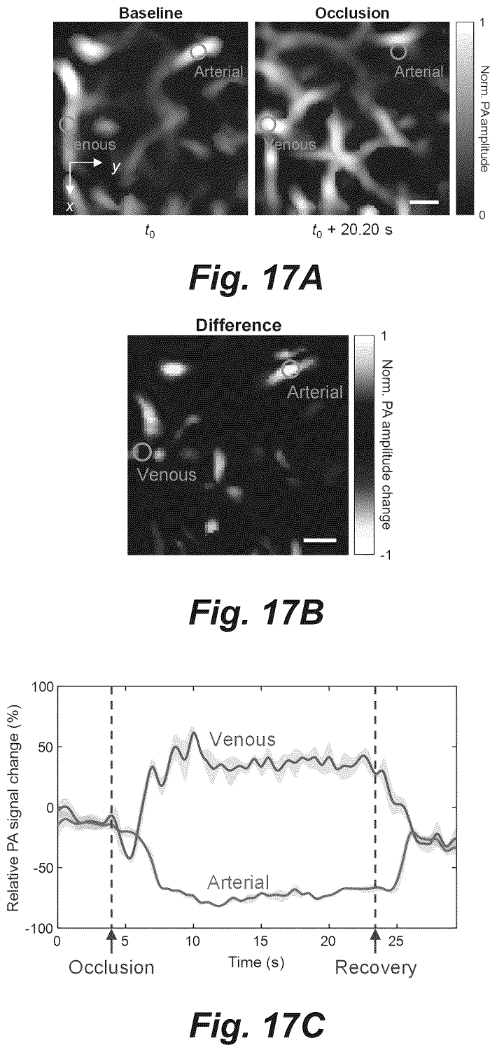

Using PACTER system 400, 3D images of blood vessels in the instep area were captured with single laser pulses both before and after vascular occlusion and the 4D dynamics of the blood vessels in response to the occlusion was reconstructed. FIG. 16B depicts a 3D PACTER image of the foot vessels of the subject before vascular occlusion (baseline). FIG. 16C depicts a 3D PACTER image of the foot vessels of the subject after vascular occlusion. FIG. 17A depicts maximum amplitude projections of the 3D volumes from the 4D PACTER datasets along the z axis in FIG. 16B. FIG. 17B depicts an illustration of a difference between the two images in FIG. 17A.

The maximum amplitude projections of the 3D volumes before and after vascular occlusion shown in FIG. 17A their difference shown in FIG. 17B showed that the blood vessels exhibited increased and decreased PA amplitudes, which suggest their roles as venous and arterial, respectively. As the occlusion progressed, the cuff prevented blood from leaving the foot via the veins, while blood continued to flow into the foot, causing the PA signals to increase in the veins (shown in blue). Conversely, the cuff caused a reduction in the blood volume and diameter of the arteries due to the gradual closing, leading to a decrease in the PA signals in the arteries (shown in red). This resulted in observable changes in the PA signals in the veins and arteries due to the occlusion preventing the blood from both entering and leaving the foot. FIG. 17C depicts a plot of relative PA signals of the venous and arterial regions indicated by the circles in FIGS. 17A and 17B respectively. The arrows and vertical lines indicate the start times of occlusion and recovery. Upon release of the cuff, the PA signals in the arteries increased, indicating the acute opening of the arteries with the full release of the blood flow, while the PA signals in the veins decreased. This process, known as post-occlusive reactive hyperemia, is consistent with previous results.

These results demonstrate the potential for PACTER techniques being a tool for assessing vascular function in the lower extremities. The detailed and localized information PACTER techniques provide could prove invaluable in early-stage screening, leading to more sensitive detection or early prevention of conditions such as ischemia or ulcer development. Moreover, with PACTER's ability to accurately monitor hemodynamic changes, PACTER techniques could facilitate the measurement of treatment efficacy for peripheral vascular diseases and diabetes. PACTER techniques could prove valuable for guiding wound treatment in vascular clinics and could facilitate post-surgical decision-making and provide longitudinal monitoring of functional wound healing. Ultimately, PACTER techniques hold substantial promise for enhancing patient outcomes and advancing medical research in peripheral vascular diseases and diabetes.

(E) Comparison with Other Techniques that Use Other Ergodic Relays

Table 1 is a comparison of PACTER techniques of certain embodiments with PA topography through an ER (PATER) and PA microscopy through an ER (PAMER) that use other types of ergodic relays. First, other techniques only allow for 2D imaging and do not provide depth information about the object. PACTER techniques enables both 3D and 4D in vivo imaging in animals and humans. Second, other techniques are not suited for long-term imaging in unstable environments. For example, PA topography through an ER (PATER) and PA microscopy through an ER (PAMER) are sensitive to boundary condition between the object and the ER, necessitating recalibration for different objects. PACTER techniques employs an integrated transducer that is directly fabricated on the ER, enhancing sensitivity over a broadband while enabling long-term in vivo imaging in unstable environments. Third, all other techniques employ a two-step iterative shrinkage/thresholding (TwIST) algorithm for reconstruction, which can be orders of magnitude slower than the Fast Iterative Shrinkage-Thresholding Algorithm (FISTA) algorithm used in PACTER techniques. An example of the FISTA algorithm may be found in Beck, A. & Teboulle, M. A, “fast iterative shrinkage-thresholding algorithm for linear inverse problems, SIAM,” Imaging Sci. 2, 183-202 (2009). For the substantial number of voxels 80×80×120 in the 3D volumes that may be imaged by PACTER techniques, the reconstruction becomes computationally intensive. PACTER reconstruction may employ FISTA and temporal convolution implemented through fast Fourier transform (FFT)f or 3D and 4D imaging through ERs to improve the computations. Finally, due to the issues associated with other techniques that use other ergodic relays, they may not be able to provide human imaging. In comparison, PACTER techniques have the ability to capture dynamic changes in vascular occlusion which offers clinical potential for early detection, enhanced assessment, and personalized treatment of peripheral vascular diseases.

TABLE 1

| | PATER | PAMER | PACTER |
|---|---|---|---|
| Universal calibration | No | No | Yes |
| Long-term stability | No | No | Yes |
| Single-shot 2D imaging | Yes | No | Yes |
| Single-shot 3D imaging | No | No | Yes |
| 4D imaging | No | No | Yes |
| Thin object requirement | No | No | No |
| Transducer coupling | Post- | Post- | Integrated |
| | fabrication | fabrication | (metallic) |
| | (resin) | (resin) | |
| Transducer sensitivity | Low | Low | High |
| Reconstruction algorithm | TwIST | TwIST | FISTA & FFT |
| Animal imaging | Yes | Yes | Yes |
| Human imaging | No | No | Yes |
| Clinical applicability | No | No | Yes |

### (F) Some Examples of Alternative System Configurations/Components

Although certain implementations of a PACTER system employ one or more motorized stages for calibration, a pulsed laser for illumination, and a DAQ card for data acquisition, other components may be used for less expensive and compact alternative implementations. For example, given the system's universal calibration capability and the fact that a single calibration dataset could remain effective for at least a year as discussed in Section II (A) with respect to FIG. 26, the ergodic relay in a PACTER system could be pre-calibrated, eliminating the need for motorized stages during the system's distribution. As another example, the pulsed laser and/or or DAQ card may be replaced with light-emitting diodes (LEDs) and one or more microcontrollers, respectively, which could reduce cost and further enhance the portability of the system. Some examples of suitable LEDs may be found in Zhu, Y. et al., “Light Emitting Diodes based Photoacoustic Imaging and Potential Clinical Applications,” Sci. Rep. 8, 9885 (2018). Some examples of suitable microcontrollers may be found in Fatima, A. et al., “Review of cost reduction methods in photoacoustic computed tomography,” Photoacoustics 15, 100137 (2019). Additionally, mass production of the ER and the single-element ultrasonic transducer could substantially lower the cost of the system, making PACTER more accessible in low-resource settings, further lowering the barriers to clinical translation.

### (1) Handheld Configuration

In certain embodiments, a PACTER system is configured for handheld operation. Handheld operation offers the flexibility for imaging different body parts in both animals and humans. In some cases, a PACTER system includes one or more optical fibers to enable handheld operation. For example, a PACTER system may implement a multi-mode fiber to deliver a light beam, which may add flexibility for animal and human imaging.

FIG. 29 is a schematic drawing of a PACTER system 2900 configured for handheld imaging operation, according to an embodiment. PACTER system 2900 includes a housing 2992 and an ergodic relay 2950 with a prism 2952 optically coupled (e.g., fused) to an optical rod 2954 and an integrated single-element transducer 2990. Ergodic relay 2950 is at least partially located within housing 2992. PACTER system 2900 also includes a temperature stabilizing system including a temperature stabilizing box 2990 around at least a portion of an exterior of ergodic relay 2950. In one implementation, the entire ergodic relay is sealed within the temperature stabilizing box 2990.

PACTER system 2900 also includes a multi-mode optical fiber 2912 coupled to a fiber collimator 2911, which is in optical communication with prism 2952. In this embodiment, the pulsed light source is a separate component and one or more laser pulses are delivered to the prism 2952 via the multi-mode optical fiber 2912. A homogenizer may also be a separate component. Although not shown, PACTER system 2900 includes additional system components such as, e.g., data acquisition system or one or more microcontrollers and an optical system. A computing device may also be a separate component. Photoacoustic data may be communicated to the computing device via a communication cable or in wireless form.

### (2) Temperature Control System

Due to the large dimensions of the ergodic relay as compared with the acoustic wavelength, the photoacoustic waves need to propagate a long distance inside the ergodic relay. A slight change in the speed of sound due to temperature fluctuations=might cause large differences in a measured PACTER signal. Certain implementations employee a temperature control system with one or more elements for maintaining the temperature of the ergodic relay, e.g., at 30° C., to maintain a constant speed of sound during operation. For example, in one implementation, a PACTER system includes a temperature stabilizing box to maintain the temperature of the ergodic relay.

FIG. 30A is a schematic drawing of a PACTER system 3000 with a temperature control system, according to an embodiment. PACTER system 3000 includes a housing 3092 and an ergodic relay 3050 with a prism 3052 optically coupled to an optical rod 3054 and an integrated single-element transducer 3090. The ergodic relay 3050 is partially located within housing 3092. The temperature control system includes a temperature stabilizing box 3090, a thermocouple 3091 coupled to the optical rod 3054, a temperature controller 3094 in electronic communication with thermocouple 3091 to receive temperature data, and a heating element 3093 such a heating pad. The temperature stabilizing box 3090 lies at least around an exterior of the ergodic relay 3050. PACTER system 3000 may also include one or more optical fibers for delivering one or more light pulses to the prism 3052. A homogenizer may also be a separate component. Although not shown, PACTER system 3000 includes additional system components such as, e.g., data acquisition system or one or more microcontrollers and an optical system. A computing device may also be a separate component. Photoacoustic data may be communicated to the computing device via a communication cable or in wireless form.

FIG. 30B is a schematic drawing of a PACTER system 3001 for showing the temperature dependence, according to an embodiment. PACTER system 3001 includes an ergodic relay 3070 with a prism 3071 optically coupled to an optical rod 3072 and an integrated single-element transducer 3094. During acquisition of PACTER signals, heating element 3094 (e.g., hot air gun) is turned ON and OFF to provide periodic heating to the ergodic relay 3070.

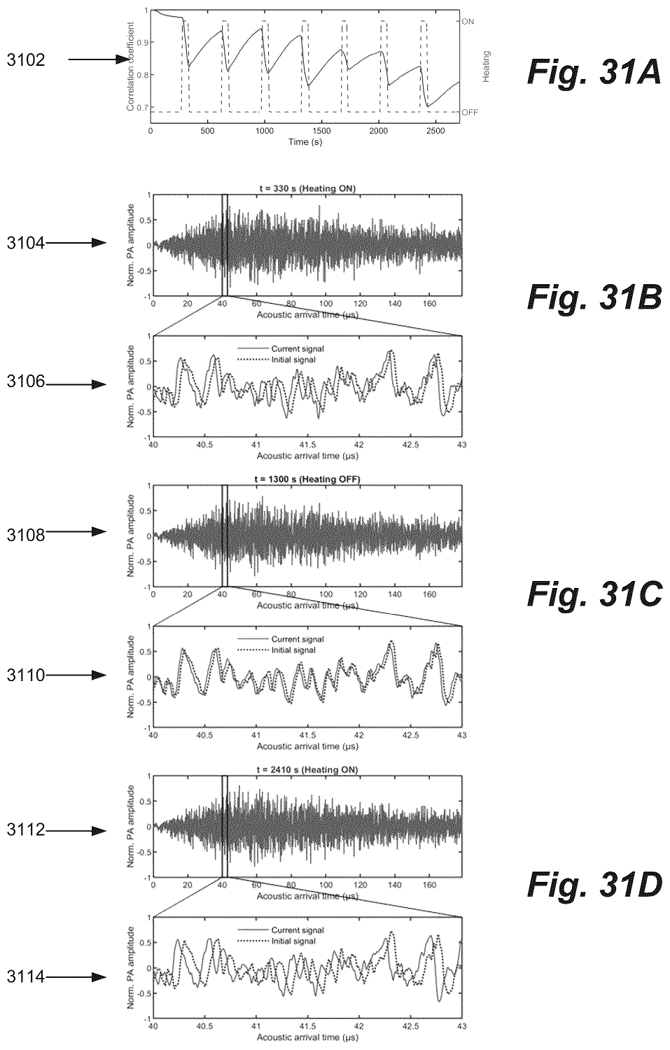

FIG. 31A depicts a graph 3102 of a correlation coefficient (solid curve) between each PACTER signal and the initial signal acquired using bovine blood, and the ON and OFF status (dashed curve) of the heating element 3094. FIG. 31B depicts a plot 3104 of a PACTER signal acquired at 330 s with heating and a plot 3106 of a zoomed-in-view of the black box in plot 3104. FIG. 31C depicts a plot 3108 of a PACTER signal acquired at 1300 s with heating off and a plot 3110 of a zoomed-in-view of the black box in plot 3108. FIG. 31D depicts a plot 3112 of a PACTER signal acquired at 2410 s with heating on and a plot 3114 of a zoomed-in-view of the black box in plot 3112.

### (3) Wavelengths

According to various implementations, the light source may provide various wavelengths. In one example, the wavelength may be in the range of 400 nm to 1064 nm. In one example, the wavelength of a laser light pulse is 532 nm. The strong attenuation of 532 nm light by endogenous chromophores in biological tissue can limit the penetration depth of the PACTER system to 3.6 mm in vivo. In certain implementations, a wavelength of 1064 nm or larger is used to increase the penetration depth to several centimeters.

### (4) Ergodic Relay Dimensions

The field-of-view of an ergodic relay with an optical rod is based on the diameter of the optical rod. In some cases, the dimensions of the prism and optical rod are selected based on: (1) the rod's diameter, which determines the FOV, should be substantially smaller than the right-angle edge length of the prism to ensure that the PA signals can be effectively scrambled; In one example, the rod's diameter can be 10 mm to 26 mm, whereas the right-angle edge length of the prism can be 40 mm. (2) the rod's length should greatly exceed the right-angle edge length of the prism to extend the duration of the object-independent signal (unaffected by the boundary condition), thereby ensuring the universal calibration capability. In one example, the rod's length can be 125 mm to 225 mm, whereas the right-angle edge length of the prism can be 20 mm to 40 mm. In certain implementations, a PACTER system may have an ergodic relay with dimensions designed for a large field-of-view, which may enable new applications such as vascular biometrics. In one example, the rod's diameter can be extended to 100 mm, allowing a field-of-view of 70×70 mm².

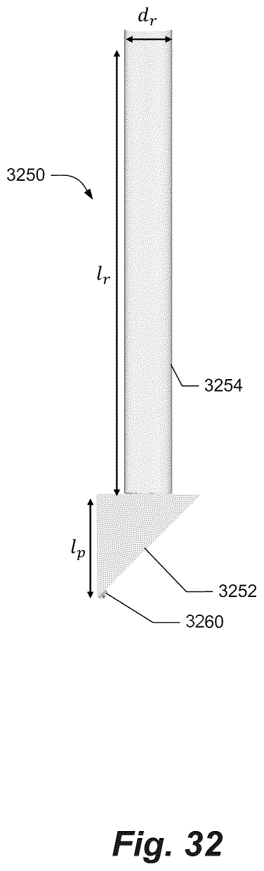

FIG. 32 depicts an ergodic relay 3250 showing the dimensions, according to an embodiment. The ergodic relay 3250 includes a prism 3252, a fused silica rod 3254, and an integrated single-element ultrasonic transducer 3260. The dimensions include the diameter (dᵣ) and the length (lᵣ) of the fused silica rod 3254, and the right-angle edge length (lₚ) of the prism 3252.

### (5) GPU

In one embodiment, the PACTER reconstruction instructions are implemented on a GPU. The 3D reconstruction in PACTER may be computationally intensive. For example, image reconstruction for a 3D volume of 80×80×120 voxels may take 10 min when using a CPU. Implementing the reconstruction instructions on a GPU may significantly improve computational time.

### (6) Acoustic Impedance-Matching Layer(s)

An acoustic impedance mismatch between the object and the ergodic relay can cause a limited-view effect that limits spatial resolution. In certain embodiments, the ergodic relay of a PACTER system includes one or more acoustic impedance-matching layers disposed between the object being imaged and the ergodic relay, e.g., on a distal end of an optical rod of the ergodic relay. For example, a quarter-wavelength impedance-matching layer (Z_{match}=\sqrt{Z_{object} Z_{ER}}) may be implemented where Z_{object} and Z_{ER} are the acoustic impedances of the object and the ergodic relay, respectively. As another example, a cascaded impedance-matching layer may be used. As another example, a cascaded impedance-matching layer may be used. As another example, a gradient impedance-matching layer may be used. Some examples of suitable acoustic impedance-matching layers may be described in Zhao, J. et al., “Ultrawide Bandwidth High-Frequency Ultrasonic Transducers With Gradient Acoustic Impedance Matching Layer for Biomedical Imaging,” IEEE Trans. Ultrason. Ferroelectr. Freq. Control 69, 1952-1959 (2022), Zhao, J. et al., “Ultrawide Bandwidth High-Frequency Ultrasonic Transducers With Gradient Acoustic Impedance Matching Layer for Biomedical Imaging,” IEEE Trans. Ultrason. Ferroelectr. Freq. Control 69, 1952-1959 (2022)), and Li, Z. et al. Broadband gradient impedance matching using an acoustic metamaterial for ultrasonic transducers. Sci. Rep. 7, 42863 (2017). Some examples of materials of an acoustic impedance-matching layer include polymethyl methacrylate and polymethylpentene.

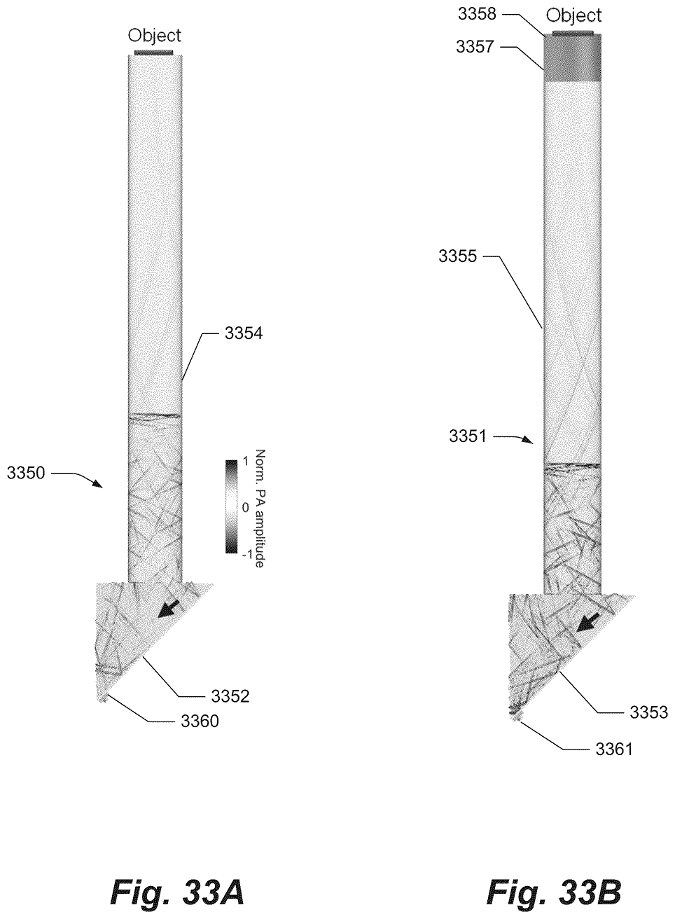

FIG. 33A depicts a schematic diagram of an ergodic relay 3350 in a PACTER system showing a simulated PA signal propagating in the ergodic relay 3350, according to an embodiment. Ergodic relay 3350 includes a prism 3352, an optical rod 3354, and an integrated single-element ultrasonic transducer 3360.

FIG. 33B depicts a schematic diagram of ergodic relay 3351 with an acoustic impedance-matching layer 3357, according to an embodiment. Ergodic relay 3351 includes a prism 3353, an optical rod 3355, and an integrated single-element ultrasonic transducer 3361 on an outer surface of prism 3353. Acoustic impedance-matching layer 3357 is at a distal end of optical rod 3355. Acoustic impedance-matching layer 3357 is located between the object being imaged and optical rod 3355. The acoustic impedance-matching layer 3357 may be, e.g., deposited an object surface 3358 at a distal end of the optical rod 3355. A quarter-wavelength matching layer was used in the illustrated simulation. A simulated PA signal is shown propagating in ergodic relay 3351. Black arrows in FIGS. 33A and 33B show that the PA signal in FIG. 33B is stronger than the PA signal in FIG. 33A. The acoustic impedance-matching layer may be, for example, (1) a quarter-wavelength impedance-matching layer (Z_{match}), where Z_{object} and Z_{ER} are the acoustic impedances of the object and the ergodic relay 4251, respectively, (2) a cascaded impedance-matching layer, and (3) a gradient impedance-matching layer.

### (7) Other Applications

PACTER may impact a wide range of applications in biomedical research and clinical settings, including home care of diabetic-foot ulcers or peripheral vascular diseases, point-of-care screening for hypertension, and simultaneous oximetry of both arterial and venous blood in intensive care units. PACTER's single-shot volumetric imaging concept using a single-element detector can extend beyond optical imaging, aiding fields such as medical ultrasonography, underwater sonar, and airborne radar. For instance, PACTER can potentially replace the transduce array for detection in ultrasonography, thereby substantially reducing the system's cost and complexity.

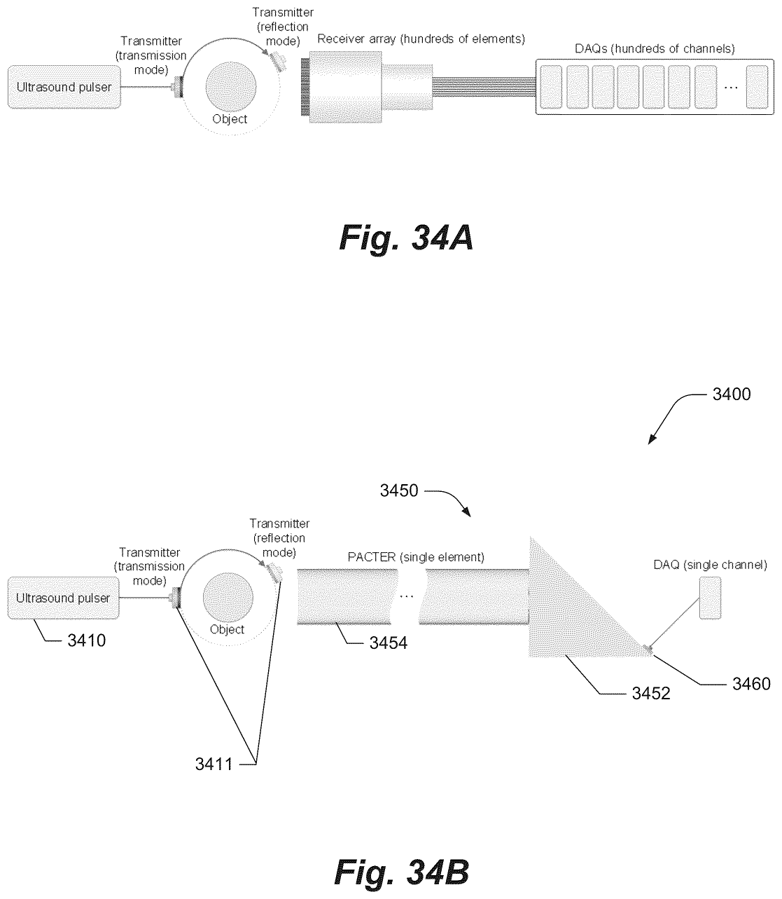

FIG. 34A depicts a schematic drawing of an ultrasonography system with a transmitter and a receiver array with hundreds of elements, which requires a DAQ device with hundreds of channels. The ultrasonography system includes an ultrasound pulser with a transmitter. The position of the transmitter can be adjusted to allow both transmission- and reflection-mode imaging.

FIG. 34B depicts a schematic drawing of ultrasonography PACTER system 3400 (ultrasonography system implementing PACTER technique), according to an embodiment. PACTER system 3400 includes an ergodic relay 3450 having a prism 3452, an optical rod 3454, and an integrated single-element transducer 3460, according to an embodiment. The ergodic relay 3450 may be the same as the ergodic relay 352 in FIG. 3. The PACTER system 3400 includes, or is in communication with, an ultrasound pulser 3410 with at least one transmitter 3411 for generating one or more light pulses. The position of the transmitter 3411 can be adjusted to allow both transmission- and reflection-mode imaging. Comparing with the ultrasonography system in FIG. 34A, the receiver array is replaced by an ergodic relay with a single-element transducer, which requires a DAQ device with only a single channel. The ultrasonography PACTER system 3400 can implement the calibration and imaging methods described in Section IV.

In some embodiments, the one or more light pulses are provided to the object being imaged directly from the one or more light sources or via one or more optical elements in optical communication with the one or more light sources. For example, in the ultrasonography PACTER system 3400 of FIG. 34B, the one or more light pulses are provided directly to the object by a transmitter 3411 of the ultrasound pulser 3410. It would be understood that although the PACTER systems of various examples described herein provide one or more light pulses to the acoustic cavity, in alternative implementations, the one or more light pulses may be provided to the object directly from the one or more light sources or via one or more optical elements in optical communication with the one or more light sources. For example, in an alternative embodiment, homogenizer 121 in FIG. 1 provides homogenized light directly to object 20.

In summary, PACTER techniques are noninvasive, label-free, and ultrafast imaging techniques that enable 4D imaging of hemodynamics in humans using the 1D signal captured by a single detector, achieving an imaging speed of up to a thousand volumes per second. PACTER's capability to visualize the 4D hemodynamics in humans and small animals, particularly the hemodynamic changes in human foot vessels during vascular occlusion are discussed in Section IV. PACTER techniques can image different objects, including human hands and mouse abdomens, without the need for recalibration. PACTER high imaging speed allows for immediate intervention in case of abnormal hemodynamic changes. Additionally, PACTER's low cost and compact form factor may be ideal for point-of-care testing, facilitating quick and easy assessment of hemodynamic parameters at the bedside or in remote locations.

### V. Examples of Certain PACTER System Components and Operations

### (A) Homogenizer

In certain embodiments, a PACTER system includes a homogenizer to convert light to uniform light that can be provided to the acoustic cavity of the ergodic relay. An example of a suitable homogenizer is a fly's eye homogenizer.

FIG. 22A is a schematic illustration of the working principle of a fly's eye homogenizer having a microlens array 2201 (e.g., #64-480 lens array sold by Edmund Optics), according to an embodiment. As shown, microlens array 2201 can generate a wider homogenized illumination pattern at the imaging plane. FIG. 22B depicts a first photograph 2211 of the spatial distribution of an original laser beam from a high power laser source and a second photograph 2212 of a homogenized beam.

FIG. 22C depicts a schematic illustration of an example of an optical path light through a fly's eye homogenizer 2234 and an ergodic relay 2250 of a PACTER system 2200, according to an embodiment. The ergodic relay 2250 includes a right-angle silica prism 2252 fused to a silica rod 2254 and an ultrasonic transducer element 2660 disposed on a surface of right-angle silica prism 2252. The fly's eye homogenizer 2234 includes a first microlens array 2235 (e.g., #64-480 lens array sold by Edmund Optics) and a second microlens array 2236 (e.g., #64-480 lens array sold by Edmund Optics) side-by-side. The PACTER system 2200 also includes a spherical lens 2237 (e.g., AC254-250-A lens sold by Thorlabs). The original beam 2213 passes through first microlens array 2235, second microlens array 2236, and spherical lens 2237 to form focused uniform illumination 2215 that is propagated to right-angle silica prism 2252.

As shown in FIG. 22C, the two microlens arrays 2235, 2236 form multiple parallel Köhler illumination systems side-by-side. The original beam 2213 entering first microlens array 2235 is divided into multiple beamlets. Through the lenslets pairs in microlens arrays 2235, 2236 and spherical lens 2237, each beamlet of the original beam 2213 is imaged to the homogenization plane, i.e., the top of the ergodic relay 2250. Because the images of the beamlets are all superimposed on the homogenization plane, the intensity differences among the beamlets disappear in their superimposed images. Therefore, the intensity distribution of the homogenized beam 2614 is independent of the homogeneity of the original beam 2213. Further, the square-type microlens arrays in the setup will generate a square flat-top intensity distribution in the homogenization plane, which provides a good match for the square-type calibration pattern in PACTER techniques, allowing accurate mapping between the calibration and imaging areas.

The width of the homogenized beam, d_{H}, is given by:

$${d}_{H} = \frac{{p}_{M} {f}_{L} ( 2 {f}_{M} - a )}{{f}_{M}^{2}} , \qquad ( Eqn . 4 )$$

where p_{M} and f_{M} are the pitch and focal length of the lenslets in the two identical microlens arrays, f_{L} is the focal length of the spherical lens, and a is the separation between the microlens arrays. In PACTER techniques, a is set to be identical to f_{M}, leading to:

$${d}_{H} = \frac{{p}_{M} {f}_{L}}{{f}_{M}} . \qquad ( Eqn . 5 )$$

The divergence half-angle after the homogenization plane, θ, is given by:

$$θ = atan ( \frac{{d}_{O} + {d}_{H} - {p}_{M}}{2 {f}_{L}} ) , \qquad ( Eqn . 6 )$$

where d_{O} is the diameter of the original beam.

In one example, a PACTER system implements a microlens arrays with p_{M}=0.5 mm and f_{M}=15 mm, a spherical lens with f_{L}=250 mm, and an original beam with a diameter of d_{O}=6 mm. Therefore, the homogenized beam has a width of d_{H}≈8 mm, matching the size of the calibration pattern (80 by 80 steps with a step size of 0.1 mm), and the divergence half-angle θ≈2°. The small divergence ensures homogenous illumination across the whole 3D volume (8 mm×8 mm×3.6 mm) for in vivo imaging. Within the 3.6 mm depth, the illumination beam merely diverges laterally by 0.13 mm, which is much smaller than the lateral resolution (0.56 mm) of the PACTER system. Hence, the beam divergence within the imaging volume may be ignored.

### (B) Universal Calibration of PACTER Systems

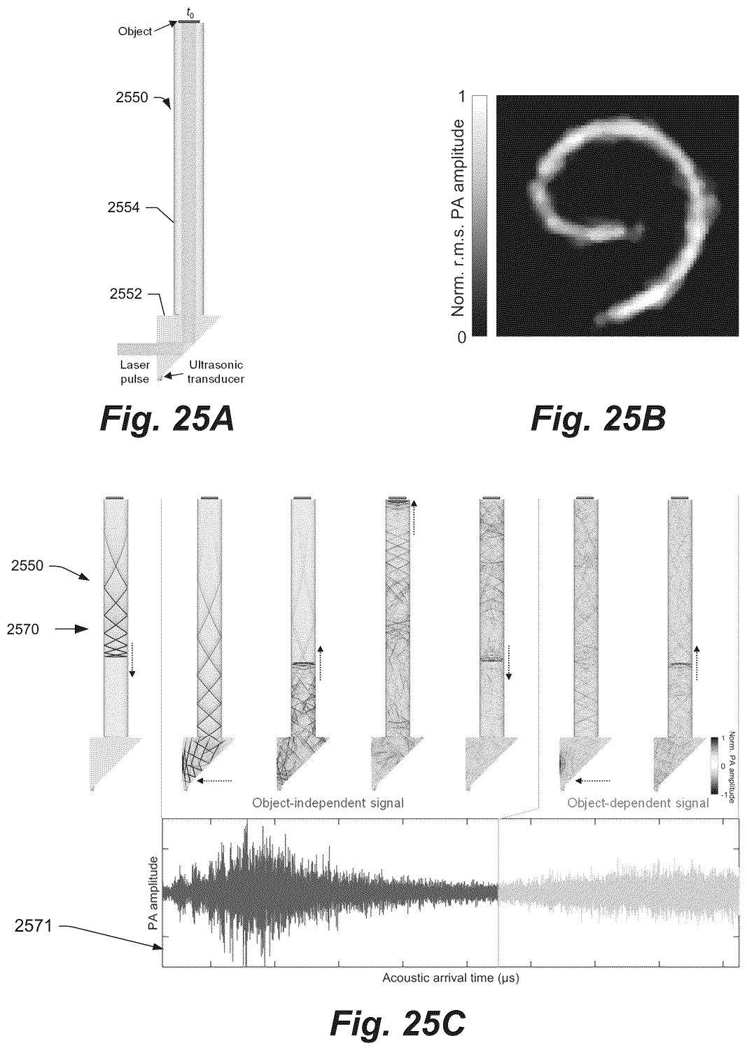

In some embodiments, a PACTER system is universally calibratable. FIG. 25A depicts a schematic drawing of an examples of an optical path of an ergodic relay 2550 of a PACTER system. The ergodic relay 2550 includes a prism 2552 fused to an acoustic delay line 2554. FIG. 25C depicts a time-lapse simulation 2570 of a PA signal propagating in the ergodic relay 2550 in the PACTER system. Black dotted arrows denote the propagating direction of the acoustic wavefront. t₀ denotes the time instance of the laser pulse illumination. t₁ denotes the time instance when the transducer starts to detect the PA signal. t₂ denotes the time instance when the transducer starts to detect the object-dependent PA signal, which is reflected from the object. FIG. 25C also depicts a plot 2571 of the PA signal detected by a transducer attached to the ergodic relay 2550 in the PACTER system. FIG. 25B is a reconstructed image of a black wire using the ergodic relay 2550 in the PACTER system calibrated with bovine blood, showing the object independence or universality of calibration.

Ergodic relay 2550 of a PACTER system includes a prism 2552 and a fused silica rod 2554, where the silica rod 2554 functions as an acoustic delay line that temporally separates the initial and reflected PA signals as shown in FIG. 30C. When a photoacoustic (PA) signal is generated from the object (t₀) and detected by the ultrasonic transducer (t₁), a part of the acoustic signal is trapped and scrambled in the prism 2552, whereas the other part is reflected toward the object and then reverberated to the transducer (t₂), as denoted by the black dotted arrows in the time-lapse simulation 2570 in FIG. 30C. Because only the reflected part of the acoustic signal will be affected by the boundary condition between the object and the ergodic relay 2550, once this part is excluded from the measurement, the acquired signal will be object independent. Consequently, the ergodic relay 2550 in a PACTER system can be used to image any object, e.g., a black wire, despite that it has been calibrated only once using a different object, e.g., bovine blood. A large segment (>100 μs) of the PACTER signal is object independent as shown in plot 2571 in FIG. 30C, enabling universal calibration in PACTER despite its 3D imaging capability.

### (C) Forward Model and Image Reconstruction

During a calibration procedure, calibrations at pixels on a 2D plane are performed. These calibration pixels can then be used as virtual ultrasonic transducers for 3D imaging. If non-zero initial pressure exists only on the calibration plane, the detected signal s(t) at time t can be expressed as:

$$s ( t ) = {\sum }_{n = 1}^{N} {p}_{n} {k}_{n} ( t ) , \qquad ( Eqn . 7 )$$

$$t \geq 0 ,$$

where N is the number of calibrated virtual transducers, kₙ(t) is the normalized impulse response from the calibration at the n-th virtual transducer, and pₙ, is the root-mean-squared PA amplitude proportional to the initial pressure at the n-th virtual transducer.

For initial pressure in a 3D volume, it is assumed M source points located at r′ₘ, m=1,2, . . . , M, in an acoustically homogeneous 3D region attached to the calibration plane. The PA wave generated from the source point at r′ₘ propagates to the calibrated virtual transducer rₙ with the speed of sound c after time

$${t}_{m , n} = \frac{‖ {r}_{m}^{′} - {r}_{n} ‖}{c} ,$$

which, through the ER, adds

$${p}_{m , n} {k}_{n} ( t - \frac{‖ {r}_{m}^{′} - {r}_{n} ‖}{c} )$$

to the detected signal, with the PA amplitude p_{m,n} quantified as

$${P}_{m , n} = \frac{w ( {θ}_{m , n} ) {p}_{0 , m}}{‖ {r}_{m}^{′} - {r}_{n} ‖} .$$

Here, θ_{m,n} denotes the incidence angle satisfying

$$cos {θ}_{m , n} = \frac{( {r}_{m}^{′} - {r}_{n} ) \cdot n}{‖ {r}_{m}^{′} - {r}_{n} ‖}$$

with n being the normal vector of the calibration plane; function w(θ_{m,n}) describes a virtual transducer's angle-dependent sensitivity; and P_{0,m} is proportional to the initial pressure at r′ₘ. pₙkₙ(t) is replaced in Eqn. 7 with

$${p}_{m , n} {k}_{n} ( t - \frac{‖ {r}_{m}^{′} - {r}_{n} ‖}{c} )$$

from all the M source points and the detected widefield PA signal is obtained:

$$s ( t ) = {\sum }_{n = 1}^{N} {\sum }_{m = 1}^{M} \frac{w ( {θ}_{m , n} ) {p}_{0 , m}}{‖ {r}_{m}^{′} - {r}_{n} ‖} {k}_{n} ( t - \frac{‖ {r}_{m}^{′} - {r}_{n} ‖}{c} ) , \qquad ( Eqn . 8 )$$

$$t \geq 0.$$

Here, define kₙ(t)=0, n=1,2 . . . , N, t<0. For sufficiently small virtual ultrasonic transducers, it is assumed that:

$$w ( {θ}_{m , n} ) = {1}_{[ 0 , {θ}_{1} ]} ( {θ}_{m , n} ) cos {θ}_{m , n} . \qquad ( Eqn . 9 )$$

Here, the following indicator function is used:

$${1}_{A} ( x ) = \{ \begin{matrix}1 , x ∈ A \\ 0 , x ∉ A\end{matrix} \qquad ( Eqn . 10 )$$

to rejection detections with incidence angles greater than the critical angle θ₁ which is quantified in Section V(D). Substituting Eqn. 9 into Eqn. 8 yields

$$s ( t ) = {\sum }_{n = 1}^{N} {\sum }_{m = 1}^{M} {p}_{0 , m} \frac{{1}_{[ 0 , {θ}_{1} ]} ( {θ}_{m , n} ) cos {θ}_{m , n}}{‖ {r}_{m}^{′} - {r}_{n} ‖} {k}_{n} ( t - \frac{‖ {r}_{m}^{′} - {r}_{n} ‖}{c} ) , \qquad ( Eqn . 11 )$$

$$t \geq 0.$$

L is set to the number of time points after temporal discretization. Then the computational complexity of a forward model based on Eqn. 11 is O(MNL).

To accelerate the forward model in Eqn. 11, the delay term

$$\frac{‖ {r}_{m}^{′} - {r}_{n} ‖}{c}$$

is split from function kₙ(t) through temporal convolution:

$${k}_{n} ( t - \frac{‖ {r}_{m}^{′} - {r}_{n} ‖}{c} ) = δ ( t - \frac{‖ {r}_{m}^{′} - {r}_{n} ‖}{c} ) * {k}_{n} ( t ) , \qquad ( Eqn . 12 )$$

$$m = 1 , 2 , … , M ,$$

$$n = 1 , 2 , … , N ,$$

$$t \geq 0.$$

Substituting Eqn. 12 into Eqn. 11, the following is obtained:

$$s ( t ) = {\sum }_{n = 1}^{N} {k}_{n} ( t ) * {\sum }_{m = 1}^{M} {p}_{0 , m} \frac{{1}_{[ 0 , {θ}_{1} ]} ( {θ}_{m , n} ) cos {θ}_{m , n}}{‖ {r}_{m}^{′} - {r}_{n} ‖} δ ( t - \frac{‖ {r}_{m}^{′} - {r}_{n} ‖}{c} ) , \qquad ( Eqn . 13 )$$

$$t \geq 0.$$

The inner summation in Eqn. 13 has a complexity of O(MN) and each temporal convolution is implemented through three fast Fourier transforms (FFTs) with a complexity of O(Llog₂L). Thus, the forward model based on Eqn. 13 has a computational complexity of max{O(MN), O(NLlog₂L)}.

Numerical simulations were performed to quantify the improvement of computational efficiency brought by the fast algorithm. Considering that the complexities of both the slow (Eqn. 11) and fast (Eqn. 13) algorithms are linearly dependent on the number of virtual detectors N, the problem is simplified to a single virtual detector (N=1) with, e.g., L=65,536 and M=80×80×120, and only consider the computation time of a forward simulation. In a Windows 11 Home system with Intel® Core™ 19-10900T CPU @ 1.90 GHz, single-CPU-core forward simulations were performed based on the slow and fast algorithms, respectively, 36 times in Matlab. The average computation times of the simplified forward simulation are 20 s and 2.2×10⁻³ s (×9, 100 acceleration), respectively, which correspond to 35.6 h and 14.1 s for a true forward simulation (N=80×80).

Modifications, additions, or omissions may be made to any of the above-described embodiments without departing from the scope of the disclosure. Any of the embodiments described above may include more, fewer, or other features without departing from the scope of the disclosure. Additionally, the steps of described features may be performed in any suitable order without departing from the scope of the disclosure. Also, one or more features from any embodiment may be combined with one or more features of any other embodiment without departing from the scope of the disclosure. The components of any embodiment may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C #, C++ or Python, Lab VIEW, Mathematica, or other suitable language/computational software, including low level code, including code written for field programmable gate arrays, for example in VHDL. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random access memory (RAM), a read only memory (ROM), a magnetic media such as a hard-drive or a floppy disk, or an optical media such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed embodiments have been described in some detail to facilitate understanding, the described embodiments are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

What is claimed is:

1. An ergodic relay, comprising:
an acoustic delay line configured to temporally separate one or more initial photoacoustic signals and one or more reflected photoacoustic signals;
an acoustic cavity coupled to the acoustic delay line; and
an integrated single-element ultrasonic transducer fabricated onto a surface of the acoustic cavity.

2. The ergodic relay device of claim 1, wherein the acoustic delay line is an optical rod or an acoustic waveguide.

3. The ergodic relay device of claim 1, wherein the acoustic cavity is a right-angle prism.

4. The ergodic relay device of claim 3, wherein the integrated single-element ultrasonic transducer is fabricated onto the surface along a hypotenuse side of the right-angle prism.

5. The ergodic relay device of claim 1, wherein the integrated single-element ultrasonic transducer is fabricated in a region of the outer surface of the acoustic cavity that is clear from interference from an illumination beam.

6. The ergodic relay device of claim 1, wherein the acoustic delay line is a silica rod and the acoustic cavity is a glass prism fused to the silica rod.

7. The ergodic relay device of claim 1, further comprising one or more acoustic impedance-matching layers located on a distal end of the acoustic delay line.

8. A photoacoustic computed tomography system, comprising:
a housing;
an ergodic relay at least partially located within the housing, the ergodic relay comprising
an acoustic delay line configured to temporally separate one or more initial photoacoustic signals and one or more reflected photoacoustic signals,
an acoustic cavity coupled to the acoustic delay line, and
an integrated single-element ultrasonic transducer fabricated onto a surface of the acoustic cavity; and
one or more optical fibers coupled to the acoustic cavity, wherein the one or more optical fibers are configured to receive an illumination beam.

9. The photoacoustic computed tomography system of claim 8, wherein the photoacoustic computed tomography system is a handheld device.

10. The photoacoustic computed tomography system of claim 8, further comprising a homogenizer configured to convert an incident beam into homogenized light, the homogenizer in optical communication with the one or more optical fibers to deliver the illumination beam with homogenized light.

11. The photoacoustic computed tomography system of claim 10, wherein the homogenizer comprises one or more microlens arrays configured side-to-side.

12. The photoacoustic computed tomography system of claim 10, wherein the homogenizer is a fly's eye homogenizer.

13. The photoacoustic computed tomography system of claim 10, further comprising a temperature control system for controlling temperature at the ergodic relay.

14. The photoacoustic computed tomography system of claim 10, further comprising one or more acoustic impedance-matching layers located at a distal end of the acoustic delay line.

15. The photoacoustic computed tomography system of claim 10, further comprising one or more optical fibers configured deliver homogenized light to the acoustic cavity.

16. The photoacoustic computed tomography system of claim 10, wherein the integrated single-element ultrasonic transducer is fabricated in a region of an outer surface of the acoustic cavity that is clear from interference from an illumination beam.

17. The photoacoustic computed tomography system of claim 10, wherein the acoustic cavity is a right-angle prism.

18. The photoacoustic computed tomography system of claim 17, wherein the integrated single-element ultrasonic transducer is located along a hypotenuse of the right-angle prism.

19. The photoacoustic computed tomography system of claim 10, wherein the acoustic delay line is a silica rod and the acoustic cavity is a glass prism fused to the silica rod.

20. The photoacoustic computed tomography system of claim 10, further comprising a data acquisition system in electronic communication with the integrated single-element ultrasonic transducer for sampling one or more photoacoustic signals.

21. A method of fabricating an ergodic relay, the method comprising:
depositing a first electrode and a second electrode onto opposite sides of a piezoelectric crystal to form an acoustic stack;
dicing acoustic stack into a piezoelectric element;
depositing a third electrode on an outer surface of a prism fused to an optical rod; and
affixing the piezoelectric element to the third electrode on the prism.

22. The method of fabricating an ergodic relay of claim 21, further comprising depositing a layer of conductive paste on the acoustic stack to form a backing layer, wherein the piezoelectric element is affixed to the third electrode on the prism using the backing layer.

23. The method of fabricating an ergodic relay of claim 21, further comprising affixing one or more wires to the piezoelectric element.

24. The method of fabricating an ergodic relay of claim 21, further comprising depositing a protective layer on the piezoelectric element.

25. The method of fabricating an ergodic relay of claim 21, depositing one or more acoustic impedance-matching layers on a distal end of the prism.

26. The method of fabricating an ergodic relay of claim 21, wherein the piezoelectric element is affixed to the third electrode in a region of the prism that is clear from interference from an illumination beam.

27. A photoacoustic computed tomography imaging method, comprising:
receiving encoded photoacoustic data based on photoacoustic signals detected by an integrated single-element ultrasonic transducer fabricated directly onto a surface of a prism of an ergodic relay, wherein the ergodic relay includes an optical rod fused to the prism, the optical rod configured to temporally separate the photoacoustic signals and the reflected photoacoustic signals; and
reconstructing one or more 3D photoacoustic images from the photoacoustic data using a plurality of calibrated virtual transducers.

28. The photoacoustic computed tomography method of claim 27, further comprising reconstructing a sequence of 3D photoacoustic images over time.

29. The photoacoustic computed tomography method of claim 27, further comprising determining a flow rate based on the sequence of 3D photoacoustic images over time.
